(19) United States
(12) Patent Application Publication
(10) Pub. No.: US 2003/0120433 A1
(43) Pub. Date: Jun. 26, 2003

(54) Methods for predicting transcription levels

(76) Inventors: Hiroki Yokota, Carmel, IN (US); Hui Bin Sun, Indianapolis, IN (US)

Correspondence Address: DANN DORFMAN HERRELL & SKILLMAN, SUITE 720, 1601 MARKET STREET, PHILADELPHIA, PA, 19103-2307, US

(21) Appl. No.: 10/274,095

(22) Filed: Oct. 17, 2002

Related U.S. Application Data
(60) Provisional application No. 60/329,961, filed on Oct. 17, 2001.

(51) Int. Cl. C12Q 1/68; G06F 19/00; G01N 33/48; G01N 33/50

(52) U.S. Cl. 702/20; 435/6

(57) ABSTRACT

Methods are provided for empirically determining transcription levels of target genes based on the number of cis-acting elements provided in untranslated regulatory regions.

This application claims priority to U.S. provisional Application, 60/329,961 filed Oct. 17, 2001, the entire contents of which are incorporated by reference herein.

Pursuant to 35 U.S.C. §202(c) it is acknowledged that the U.S. Government has certain rights in the invention described, which was made in part with funds from the National Institutes of Health, Grant Number NIHRR17012.

### FIELD OF THE INVENTION

This invention relates to the fields of molecular biology and genomic analysis. More specifically, the present invention provides methods for empirically determining expression levels of target genes based on sequence elements present in untranslated regulatory regions. The invention also provides an assay to confirm such empirical determinations.

### BACKGROUND OF THE INVENTION

Several publications are referenced in this application in numerals in parentheses in order to more fully describe the state of the art to which this invention pertains. Full citations for these references can be found at the end of the disclosure. The disclosure of each of these publications is incorporated by reference herein.

There is a growing demand to develop computational tools for extracting functional and structural significance from human genome and various expression databases generated by high-throughput technologies (1, 2). Although current computational tools for finding genes, locating their coding sequences, and predicting their functions are valuable resources, few programs allow life scientists to integrate experimentally determined mRNA levels with a distribution of regulatory DNA elements such as AP-1, PEA-3 and Sp-1 on corresponding regulatory DNA regions (3-6). Given the present difficulties in analyzing eukaryotic gene regulation, developing an interface that integrates experimental outputs with genomic sequence databases is highly desirable.

### SUMMARY OF THE INVENTION

The present invention provides an interface that integrates experimental outputs with genomic sequence databases and facilitates the performance of model-driven genome analysis.

In a preferred embodiment a method for predicting an expression level of a target gene or gene family comprises i) selecting a target gene or gene family; ii) experimentally determining the number and type of cis-acting elements and mRNA expression levels of other genes within said target gene family to obtain a first data set; and iii) applying a PROBE algorithm to said data set, thereby generating the estimated expression level of said target gene as a function of the weighed frequencies of said cis-acting elements present in the 5′ untranslated regulatory region of said target gene. Exemplary cis-acting elements include without limitation, AP1, AP2, NFY, PEA3, Sp1, TFIID, NF-kappa B, STAT, GATA1, Oct-1 and TIE.

Expression levels of a variety of target genes or gene families may be empirically determined using the methods of the invention. Suitable gene families include matrix metalloproteinases, cytokines, hormones, cyclins, growth factor receptors, growth factors, oncogenes, and transcription factors.

In an alternative embodiment of the method of the present invention, non-linear interactions regulating transcription may be assessed. In an exemplary method, the expression level of a target gene or gene family in a particular cellular state is assessed by i) selecting a target gene or gene family; ii) experimentally determining the number and type of cis-acting elements and mRNA expression levels of other genes within said target gene family in said cellular state relative to genes not in said cellular state to obtain a first data set; and iii) applying a non-linear model algorithm to said data set, thereby generating the estimated expression level of said target gene as a function of the cellular state and the weighed frequencies of said cis-acting elements present in the 5′ untranslated regulatory region of said target gene.

In yet another embodiment, the target genes or gene families to be assessed via the methods of the present invention may be present on a microarray.

Also provided is an experimental promoter competition assay which confirms the empirical data obtained using the PROBE or non-linear algorithms of the invention. An exemplary method entails ii) providing a host cell population; ii) contacting said host cell with oligonucleotides encoding cis-acting element DNA, said cis-acting elements also being present in said target genes; iii) isolating mRNA from said host cells; iv) reverse transcribing said mRNA into cDNA; v) performing polymerase chain reaction to amplify said cDNA and assessing alterations of expression levels of said target genes in the presence and absence of said oligonucleotide encoding cis-acting element DNA, altered mRNA expression levels indicating the presence of the oligonucleotide cis-acting element in the untranslated regulatory region of said target gene.

### BRIEF DESCRIPTION OF THE DRAWINGS

FIG. 1: Flowchart of the described promoter-based algorithm

FIG. 2. Map of cis-acting elements and mRNA expression patterns for 14 MMPs in example 1. (FIG. 2A) Distribution of 7 cis-acting motifs on the 500-bp upstream sequences where the indexes A through G represent AP1, AP2, NFY, PEA3, Sp1, TFIID, and TIE, respectively. The right end of the horizontal axis corresponds to a transcription initiation site. (FIG. 2B) Observed mRNA expression pattern. Using 266 squares corresponding to 14 MMPs in 19 tissue samples, the mRNA levels are illustrated in a gray-code where darker color indicates higher expression. (FIG. 2C) Predicted mRNA expression pattern using a “leave-one-out” cross-validation procedure. (FIG. 2D) Modeled mRNA expression pattern using all MMP data.

FIG. 3. 2D scaling analysis and error analysis in example 1. (FIG. 3A) 2D Euclidian representation of 19 tissue samples based on the observed MMP expression pattern. The black circles represent rheumatoid arthritis patients, and the white circles represent non-rheumatoid arthritis patients. (FIG. 3B) 2D Euclidian representation based on the predicted MMP expression pattern. (FIG. 3C) Monte Carlo simulation for model error with the randomly assigned expression levels. The mean error ± standard deviation for 10,000 cases was 22.2±2.4. The arrow indicates the true model error of 11.3 for the expression pattern illustrated in FIG. 2D. (FIG. 3D) Positive correlation between the parameter cc and mean MMP expression level for individual samples. The best-fit-line is y=2.63x+0.18 with r²=0.98.

FIG. 4. Comparison of the measured mRNA level and the predicted mRNA level in Example 2. (FIG. 4A) Measured mRNA expression in three levels: white—the level lower than ⅓, gray—the level between ⅓ and ⅔, and black—the level higher than ⅔. (FIG. 4B) Predicted mRNA expression in three levels. (FIG. 4C) Measured mRNA expression in two levels. (D) Predicted mRNA expression in two levels.

FIG. 5. Weighting matrix and estimate of active cis-acting elements in part 2 of Example I. (FIG. 5A) Diagonal components of the weighting matrix for each gene. Among 13 genes, 7 genes, MMP-1, MMP-3, MMP-9, TIMP-1, β2-microglobulin, IL-6, and PDGF-α, had a weighting factor greater than 1. This suggests that their mRNA expression pattern fits to the linear model better than the other genes such as MMP-2, MMP-14, aFGF, bFGF, TGF-β, and TNF-α. (FIG. 5B) Estimate of active cis-acting elements such as AP1, AP2, NFY, PEA3, and Sp1 for three tissue groups. Three tissue groups are: CF—chronic fibrosing patients, Control—control individuals, and DD—Dupuytrenís disease patients.

FIG. 6. Schematic illustration of the promoter competition assay. (FIG. 6A) Control without competitive cis-acting elements. (FIG. 6B) Transcriptional machinery rendered inactive with competitive cis-acting elements that bind transcription factors.

FIG. 7. Expression of MMP mRNAs under increasing shear stress. Using MH7A synovial cells, the mRNA level of MMP-1, MMP-8, and MMP-13 was determined by RT-PCR under 1-hr uniform shear stress at 0, 1, 2, 5, and 10 dyn/cm². GAPDH served as control for RT-PCR.

FIG. 8. Expression of three MMP mRNAs in an NF-κB promoter competition assay. The level of the MMP mRNAs was determined after 1-hour incubation with DNA fragments consisting of NF-κB cis-acting elements and random DNA sequences. The concentration of the DNA fragments was 0 (normal control), 0.5, 1, and 5 μM. Incubation with random DNA fragments served as negative control.

FIG. 9. Suppression of MMP-1 mRNA and MMP-13 mRNA under shear stress by NF-κB cis-acting elements. MH7A cells were incubated for 1 hour with 5 μM DNA fragments consisting of NF-κB cis-acting elements or random sequences. The cells were grown under 0 or 10 dyn/cm shear stress for 1 hour, and the levels of mRNAs corresponding to MMP-1, MMP-8, and MMP-13 was determined by RT-PCR. NC: normal control; RC: control incubated with random DNA sequences; and NF-κB: experimental incubated with NF-κB cis-acting elements.

FIG. 10. Comparison of DNA fragments with and without phosphorothioate modification. The mRNA level is normalized by the basal control level, and a standard deviation is indicated by a bar. The white, gray, and black columns correspond to the random DNA fragments, NF-κB fragments without modification, and NF-κB fragments with phosphorothioate modification. (FIG. 10A) Level of MMP-1 mRNA. (FIG. 10B) Level of MMP-13 mRNA.

FIG. 11. Alteration in mRNA levels of the 45 IL1-responsive genes. (FIG. 11A) Observed mRNA alteration. The columns marked with “−” and “+” represent the mRNA levels before and after the IL1 treatment, respectively. The color-coded column displays the logarithmic expression ratio of the IL1-induced level to the basal control level, where “red” and “green” indicates up- and downregulation, respectively. (FIG. 11B) Modeled expression pattern based on a 300-bp upstream regulatory DNA region. As a candidate for putative TF binding motifs, 512 DNA fragments 5 bp in length were considered, and the models with 1, 2, 4, 8, 16, and 32 putative TF binding motifs are illustrated.

FIG. 12. Length of the upstream regulatory DNA sequences and modeling error. The model used the optimal choice of 8 putative TF binding motifs that varied depending on length of the upstream regulatory DNA sequences. (FIG. 12A) GC contents in the selected putative TF binding motifs and the upstream regulatory DNA sequences. (FIG. 12B) Correct up/down prediction rate for the 45 IL1-responsive genes. In Monte Carlo simulation the mRNA expression level was scrambled among the 45 genes and the average of 10,000 cases was presented. (FIG. 12C) Histogram showing the correct up/down prediction rate in Monte Carlo simulation. Mean and standard deviation were 33.4 and 1.4. The arrowhead (39.8) indicates mean of the model for the upstream regulatory DNA sequences 200-1,000 bp in length.

FIG. 13. Eigengenes, eigenvalues and weighting factors for the 45 IL1-responsive genes. (FIG. 13A) Forty-five eigengenes in U obtained by singular value decomposition of the promoter matrix such as H=UΛV^{T} using the 300-bp upstream regulatory DNA sequences. (FIG. 13B) Eigenvalues, λ₁, λ₂, . . . , λ₄₅ in Λ for the 45 eignegenes. (FIG. 13C) Weighting factors used in Eq. (3) in Example III for describing the altered mRNA level of the 45 IL1-responsive genes by 45 eigengenes.

FIG. 14. Selection of putative TF binding motifs using eigenTF matrix, V^{T}. (FIG. 14A) Forty-five eigenTF vectors, V^{T}, corresponding to 45 eigengenes. Each column vector corresponds to one of the 512 DNA sequences such as AAAAA, AAAAC, etc. (FIG. 14B) Weighted eigenTF vectors using the weighting factors illustrated in FIG. 3C. (FIG. 14C) Eight putative TF binding motifs identified by the eigengene-eigenTF analysis using H=UΛV^{T}.

FIG. 15. Estimated cellular state of the 8 putative TF binding motifs. The positive value suggests the stimulatory role, and the negative value indicates the inhibitory role in the responses to IL1. Sixteen different models with 1-16 putative TF binding motifs were analyzed, and the estimated state of the first 8 putative TF binding motifs is plotted where the x-axis indicates the number of TF binding motifs used in the model. The DNA sequence of CAGGC in (FIG. 15A) was included in all models with 1-16 putative TF binding motifs, and the DNA sequence of CCGCG in (FIG. 15H) was used in the models with 9-16 putative TF binding motifs.

FIG. 16. PROCO assay for LIF, NFκB, and IRF1 expression in the presence of IL1. The three DNA fragments used in PROCO assay were: (FIG. 16A) 5′-ATCAGCAGGCATACG-3′; (FIG. 16B) 5′-ACAATCCGCCGTTTA-3′; and (FIG. 16C) 5′-ACAATCCGCCGTTTA-3′. GAPDH is used as RT-PCR control.

FIG. 17. Shear-induced expression of a family of MMP genes. (FIG. 17A) Experimentally determined mRNA expression pattern of 14 MMP genes under 2 dyn/cm² shear for 0-24 hours. The column (a-f) designates the duration under shear for 0, 1, 3, 6, 12, and 24 hours respectively. (FIG. 17B) Hierarchical clustering dendogram of 13 MMP mRNA expression profiles in response to shear stress at 2 dyn/cm².

FIG. 18. Distribution of TF binding motifs in the 5′-flanking regulatory region of a family of MMP genes. The 1000-bp upstream sequences are mapped for seven TF binding motifs such as AP1 (FIG. 18A), AP2 (FIG. 18B), NFY (FIG. 18C), NFκB (FIG. 18D), PEA3 (FIG. 18E), Sp1 (FIG. 18F) and STAT (FIG. 18G). The arrow indicates the predicted site of transcription initiation.

FIG. 19. Modeling error for the linear and nonlinear formulations. (FIG. 19A) Modeling error as a function of the length of the regulatory DNA sequences. The nonlinear model with λ=7 is based on the upstream regulatory DNA sequences 180-2000 bp in length. (FIG. 19B) Fourier analysis of the modeling error for the nonlinear formulation with λ=7. (FIG. 19C) Modeling error for the 200-bp and 730-bp regulatory DNA sequences as a function of the nonlinear parameter, λ. (FIG. 19D) Monte Carlo simulation of modeling error for the 200-bp regulatory DNA sequences. The letters, “a” and “b,” indicate the modeling error of the nonlinear and linear formulations, respectively.

FIG. 20. Expression pattern and multiscaling analysis of a family of MMP genes. In (FIGS. 20A)-(20C), the column (a-f) designates the duration under shear for 0 (control), 1, 3, 6, 12, and 24 hours respectively, and the row represents individual MMP genes including MMP1, 2, 3, 7, 8, 9, 10, 11, 12, 13, 14, 15, 16, and 20. In (D)-(F), 14 MMPs are positioned in 2D Euclidian space based on similarity of their responses to shear. (FIG. 20A) Observed expression pattern. (FIG. 20B) Modeled expression pattern using the linear formulation with the 200-bp regulatory DNA sequences, and the modeling error. (FIG. 20C) Modeled expression pattern using the nonlinear formulation with the 200-bp regulatory DNA sequences and the modeling error. The nonlinear parameter, λ, was set to 7. (FIG. 20D) 2D positioning based on the observed expression pattern of 12 MMPs. (FIG. 20E) 2D positioning based on the linear model. (FIG. 20F) 2D positioning based on the nonlinear model.

FIG. 21. Nonlinear models using 1-7 known TF binding motifs. (FIG. 21A) Experimentally determined expression pattern (identical to FIG. 19A). (FIG. 21B) Modeled pattern based on the 200-bp upstream DNA sequences using 7 (AP1, AP2, NFY, NFκB, PEA3, Sp1, STAT), 5 (AP2, NFY, PEA3, Sp1, STAT), 3 (NFY, Sp1, STAT), and 1 (Sp1) member of the TF binding sites. (FIG. 21C) Modeled pattern based on the 730-bp upstream DNA sequences using 7, 5 (AP1, AP2, NFY, Sp1, STAT), 3 (NFY, Sp1, STAT), and 1 (NFY) member of the TF binding sites.

FIG. 22. Promoter competition assay for MMP1 and MMP2. GAPDH is used as PCR control. (FIG. 22A) MMP1 expression in the presence of competitive DNA fragments consisting of AP1 sites or random control DNA sequences. The concentration of the DNA fragments was 0.2, 1, 5, and 25 μM. (FIG. 22B) MMP2 expression in the presence of competitive DNA fragments at 5 μM consisting of AP1, AP2, NFκB, PEA3 sites and their combinations. NC and RC correspond to “normal control” and “random control (using the fragments with random DNA sequences)”, respectively.

FIG. 23. Role of TF binding motifs for MMP1, MMP2, MMP3, MMP8, MMP9 and MMP13. The columns, I-VII, correspond to the mRNA expression pattern inducible by TF binding sites such as AP1, AP2, NFY, NFκB, PEA3, Sp1, and STAT, respectively. The gray-scale code ranges from black (+1; strong stimulatory role) to white (−1; strong inhibitory role). (FIG. 23A) Experimentally determined role by the promoter competition assay. (FIG. 23B) Mean-square error of prediction. The broken line indicates the predicted error using the randomly generated expression data (10,000 datasets) in Monte Carlo simulation. (FIG. 23C) Predicted role by the nonlinear model with the 200-bp regulatory DNA sequences (λ=7) and the associated error. (FIG. 23D) Predicted role by the nonlinear model with the 730-bp regulatory DNA sequences (λ=8) and the associated error.

### DETAILED DESCRIPTION OF THE INVENTION

The availability of human genome sequences provides life scientists and biomedical engineers with a challenging opportunity to develop computational and experimental tools for quantitatively analyzing biological processes. In response to a growing need to integrate experimental mRNA expression data with human genome sequences, the present invention provides a unique analysis approach referred to herein as “Promoter-Based Estimation (PROBE)” analysis. The PROBE analysis is “systems analysis” of transcriptional processes using control and estimation theories. A linear model was built in order to estimate the mRNA levels of a group of genes from their regulatory DNA sequences. The model was also used to interpret two independent datasets in skeletal tissues. The results demonstrated that the mRNA levels of a family of matrix metalloproteinases can be modeled from a distribution of cis-acting elements on regulatory DNA sequences. The model accurately predicted a stimulatory role of cis-acting elements such as AP1, NFY, PEA3, and Sp1 as well as an inhibitory role of AP2. These predictions are consistent with biological observations, and a specific assay for testing such predictions is proposed. Although eukaryotic transcription is a complex mechanism, the disclosure presented here supports the use of the described analysis for elucidating the functional significance of DNA regulatory elements.

In response to a growing need to integrate experimental mRNA data with human genome sequences, we developed a unique computational approach named “PROmoter-Based Estimation (PROBE)” analysis and conducted a “systems analysis” of mRNA expression in skeletal tissues. Previously a non-model-driven approach such as cluster analysis had been developed, where quantitative expression profiles among genes are classified into hierarchical clusters based on expressional similarity (6, 7). A computational method was then developed for discovering cis-regulatory elements responsible for each cluster (8-10). However, few works have attempted to build a holistic model suitable for performing “systems analysis” of transcriptional activities. A mathematical model would be useful to life scientists and biomedical engineers if the model could evaluate the functional role of DNA regulatory elements in growth and differentiation of various tissues.

### EXAMPLE I

### Systems Analysis of Matrix Metalloproteinase mRNA Expression in Skeletal Tissues

With the understanding that individual DNA regulatory elements can be regulated with diversity and precision, we built and evaluated a linear least-square model. Focusing on the 5′-flanking regulatory region, we first counted the number of cis-acting elements for individual genes. We then modeled the experimentally observed mRNA levels using a weighed sum of the frequency of the selected cis-acting elements. In this promoter-based model, optimal weights were determined using a standard linear estimation technique. The model was used to predict a stimulatory or inhibitory role for each cis-acting element and the specific combination of cis-acting elements that would most effectively regulate mRNA expression.

To examine the PROBE algorithm, two mRNA expression datasets in skeletal tissues were used. One dataset consisted of 14 matrix metalloproteinase (MMP) genes, an influential proteolytic enzyme that degrades collagen, and collagen-associated molecules in an extracellular matrix (11-13). Controlling MMP expression is critical in preserving or remodeling skeletal tissues (14-16). We selected a heterogeneous set of genes such as MMPs, tissue inhibitors of metalloproteinases, and growth factors involved in inflammation and degradation of skeletal tissues (17). We evaluated the effects of 5-7 cis-acting elements including AP1, AP2, NFY, PEA3, Sp1, TFIID, and TIE using the PROBE algorithm and demonstrated in the two examples that the promoter-based linear model can represent at least in part complex regulatory mechanisms.

### Materials and Methods For the Practice of Example I

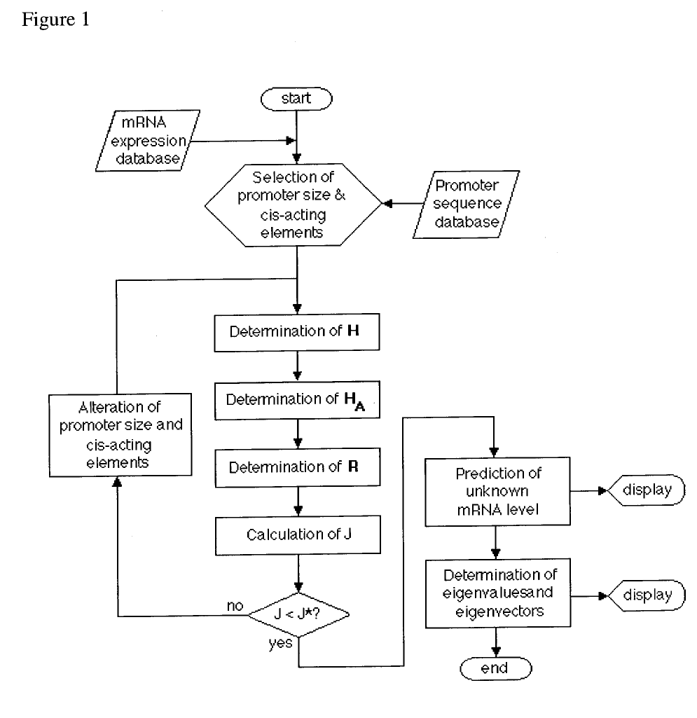

The PROBE algorithm receives two inputs such as “mRNA expression data” and “information on cis-acting DNA regulatory elements.” The linear model was built to minimize mismatches between the observed mRNA levels and the modeled mRNA levels, where three mathematical entities such as a promoter matrix (H), a promoter-associated matrix (H_{A}), and a weighting matrix (R) were defined (FIG. 1). The mathematical formulation is set forth below:

Formulation of Promoter-Based Linear Model: A transcript level of “n” genes and a level of “m” functional cis-acting elements are represented by a vector zₖ and a vector xₖ, respectively, and they are linearly linked:

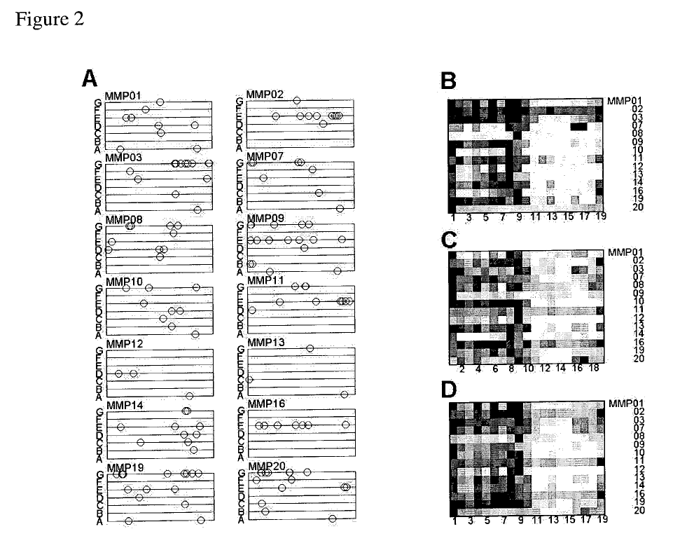

zₖ=HH_{A}xₖ+vₖ (A)

where H is an (n×m) promoter matrix, H_{A} is an (m×m) promoter-associated matrix, vₖ is a vector for measurement error, and subscript k designates tissue samples. The (i, j) component of H corresponds to the number of the j-th cis-acting element for the i-th gene. The software SIGSCAN (Version 4.05, Advance Biosciences Computing Center, University of Minnesota) was used to identify H from 5′-end regulatory regions (FIG. 2A and Table 1). H_{A} is a diagonal matrix whose j-th diagonal component weighs a contribution of the j-th cis-acting element to transcript levels. We determined the vector h_{A}=(H^{T}H)⁻¹H^{T}z^{av} and set the j-th component of h_{A} to the j-th diagonal component of H_{A}. The vector z^{av} represents the mean mRNA level among tissue samples.

TABLE 1
Promoter matrix H for Part 2 of Example 1

| | cis-acting element | | | | |
|---|---|---|---|---|---|
| gene | AP1 | AP2 | NFY | PEA3 | Sp1 |
| MMP-1 | 2 | 0 | 1 | 2 | 3 |
| MMP-2 | 0 | 0 | 0 | 1 | 18 |
| MMP-3 | 1 | 0 | 2 | 1 | 3 |
| MMP-9 | 3 | 3 | 0 | 2 | 9 |
| MMP-14 | 0 | 1 | 3 | 3 | 4 |
| TIMP-1 | 2 | 1 | 1 | 3 | 7 |
| β2-microglobulin | 0 | 0 | 1 | 2 | 7 |
| a-FGF | 0 | 1 | 1 | 2 | 6 |
| b-FGF | 1 | 1 | 0 | 0 | 20 |
| IL-1 | 1 | 0 | 2 | 2 | 8 |
| PDGF-α | 0 | 1 | 0 | 0 | 18 |
| TGF-α | 0 | 1 | 1 | 0 | 18 |
| TNF-α | 1 | 4 | 0 | 3 | 5 |

The promoter 800 bp in length was used.

In order to estimate xₖ from the observed zₖ, the function J is defined:

J=(zₖ−HH_{A}xₖ)^{T}R⁻¹(zₖ−HH_{A}xₖ) (B)

where R⁻¹ is a diagonal weighting matrix. The i-th diagonal component of R⁻¹ is set to 1/σᵢ², where σᵢ² is the approximate mean-square variation of the mRNA level for the i-th gene.

The least-square estimate of xₖ is obtained by ∂J/∂xₖ=0:

xₖ^{e}=(H_{A}^{T}H^{T}R⁻¹HH_{A})⁻¹H_{A}^{T}H^{T}R⁻¹zₖ (C)

Eigenvalue Analysis: In order to evaluate the effectiveness of the selected cis-acting elements on the mRNA expression, the eigenvalue and the eigenvector of the matrix, A=H_{A}^{T}H^{T}R⁻¹HH_{A}, was analyzed. In a linear equation such as Ay=λy, a scalar λ and a unit vector y are called an eigenvalue and an eigenvector. There are in general m sets of eigenvalues and eigenvectors corresponding to “m” cis-acting elements. In evaluating effectiveness of a particular combination of cis-acting elements for each tissue sample, we determined αₖ:

αₖ=vₗ^{T}xₖ^{e} (D)

where αₖ represents the component of xₖ^{e} parallel to the primary eigenvector. Matrix operation such as multiplication, transposition, and inversion as well as solving eigenvalues and eigenvectors were conducted using MATLAB (version 6, The Math Works, Inc.).

The multidimensional scaling analysis in 2D Euclidian space was performed using SPSS (version 11.0, LEAD Technologies, Inc.).

In order to evaluate the PROBE model, we conducted a leave-one-out cross-validation test and a Monte Carlo simulation. In leave-one-out cross-validation, the expression level of one gene in the dataset was predicted from the expression levels of the other genes. In the Monte Carlo simulation, the observed expression levels were randomly re-assigned among genes and samples, and the error estimated from 10,000 random trials were compared to the true model error for the correctly assigned expression levels.

The mRNA level of 14 MMPs including MMP-1, -2, -3, -7, -8, -9, -10, -11, -12, -13, -14, -16, -19, and -20 was obtained from the study of rheumatoid arthritis and traumatic disease conducted by Konttinen et al. (11). The extraction of mRNA was performed two or more times and the results were shown to be reproducible. The expression level was represented by a value in the range of 0 to 1 and a continuous gray level was used to illustrate the expression profile. Ten tissue samples (k=1 to 10) were derived from rheumatoid arthritis patients, and nine tissue samples (k=11 to 19) were isolated from traumatic disease patients. The MMP expression of rheumatic tissue was on average higher than that of traumatic patients.

The 5′-end upstream regulatory region, 500 bp in length, was used for the PROBE analysis. MMP-15 and MMP-17 were excluded, since the size of a dominant PCR fragment differed from the control and the promoter was not retrievable from the currently available human genome. The accession numbers were AJ002550 (MMP-1), AJ298926 (MMP-2), U51914 (MMP-3), NT009151 (MMPs-7, -10, -12, and -20), AF059679 (MMP-8), NT011375 (MMP-9), NT011520 (MMP-11), U52692 (MMP-13), NT024615 (MMP-14), NT008256 (MMP-16), and NT009458 (MMP-19). Cis-acting regulatory elements such as AP1, AP2, NFY, PEA3, Sp1, TFIID, and TIE were considered.

The mRNA expression data were obtained from the study conducted by Bunker et al. (17). The data included three sample groups such as chronic fibrosing patients, control individuals, and Dupuytren's disease patients. We focused on 13 genes such as MMP-1, MMP-2, MMP-3, MMP-9, MMP-14, TIMP-1, β-2 microglobulin, acidic fibroblast growth factor (a-FGF), basic fibroblast growth factor (b-FGF), interleukin-6 (IL-6), platelet driven growth factor-α (PDGF-α), transforming growth factor-β (TGF-β), and tumor necrosis factor-α (TNF-α).

The expression level in each sample group was defined by Nᵢₖ⁺/Nₖ, where Nᵢₖ⁺=the number of the tissue samples expressing the i-th gene in the k-th group, and Nₖ=the total number of tissue samples in the k-th group. The accession numbers were NT019712 (TIMP-1), NT010302 (β-2 microglobulin), NT016788 (a-FGF), NT016354 (b-FGF), AF869204 (IL-6), M59423 (PDGF-α), NT01 1139 (TGF-β), and NT023426 (TNF-α). Five cis-acting regulatory elements, AP1, AP2, NFY, PEA3, and Sp1, were considered in this example. The PROBE analysis is not designed to model post-transcriptional processes. Therefore, genes such as IL-1 on which the mRNA level is regulated after transcription were excluded.

### Results

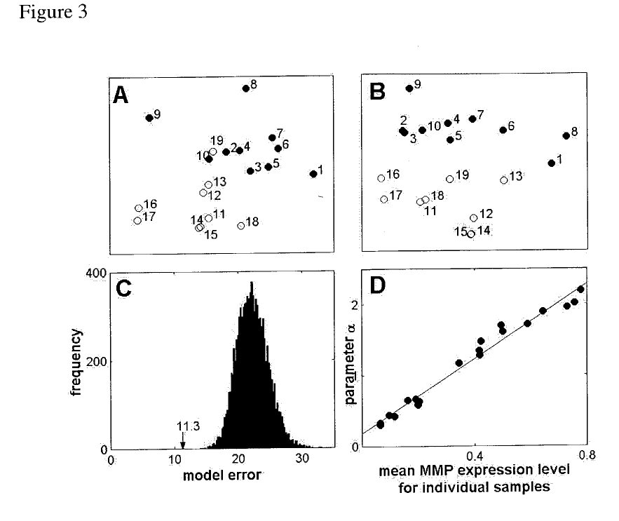

Modeling of MMP transcript levels: Using the seven cis-acting elements (AP1, AP2, NFY, PEA3, Sp1, TFIID, and TIE) on the 500-bp upstream DNA sequences, the least-square estimator was applied to the dataset consisting of the transcript levels of 14 MMPs for 19 samples (FIG. 2). Two simulated expression patterns were derived from the observed expression pattern in a continuous gray code. The expression pattern illustrated in FIG. 2C was generated by the “leave-one-out” cross-validation procedure, and the expression pattern in FIG. 2D was modeled using all available data. The multidimensional scaling analysis was performed to locate the 19 tissue samples in 2D Euclidian space, where the black and white circles represented the samples from the patients with rheumatoid arthritis and non-rheumatoid arthritis, respectively, for the observed (FIG. 3A) and predicted expression patterns (FIG. 3B). The model error, defined by Eq. (B) above, was calculated as 11.3 for the modeled pattern depicted in FIG. 2D. When the expression levels were randomly re-assigned among 266 data points, the mean error and the standard deviation were 22.2 and 2.4 for 10,000 cases in the Monte Carlo simulation (FIG. 3C).

Sensitivity analysis: We next conducted a sensitivity analysis using eigenvalues and eigenvectors as indicators where the effectiveness of each cis-acting element on MMP expression was examined. There are seven sets of eigenvalues and unit eigenvectors corresponding to the seven selected cis-acting elements. An eigenvector represents a specific combination of seven cis-acting elements and an associated eigenvalue indicates effectiveness of the combination in regulating mRNA levels. The calculated eigenvalues were 1.44, 0.35, 0.11, 0.07, 0.02, 0.004, and 0.0001, and the eigenvector corresponding to the largest eigenvalue was (0. 209, −0.209, 0.240, 0.004, 0.921, −0.055, 0.068)^{T} in the 7-dim space of AP1, AP2, NFY, PEA3, Sp1, TFIID, and TIE. The positive values in the elements of the eigenvector indicated a stimulatory role of the corresponding cis-acting elements (AP1, NFY, PEA3, Sp1, and TIE) and the negative values suggested an inhibitory role (AP2, and TFIID).

Since each skeletal tissue exhibited a unique MMP mRNA pattern, the estimate of active cis-acting elements must differ among the 19 tissue samples. In order to examine the role of the selected cis-regulatory elements, we determined a component of active cis-acting elements projected onto the eigenvector with the largest eigenvalue. In a linear estimation analysis, this projected component serves as an indicator of the MMP expression levels. Indeed, the component was positively correlated to each tissue's mean MMP expression level with a correlation coefficient of 0.98 (FIG. 3D). The average value was 0.567 and 0.169 for the tissues derived from rheumatic arthritis and traumatic diseases respectively, consistent with the observation that the rheumatic arthritis tissues present a higher level of MMP mRNAs than the traumatic disease tissues.

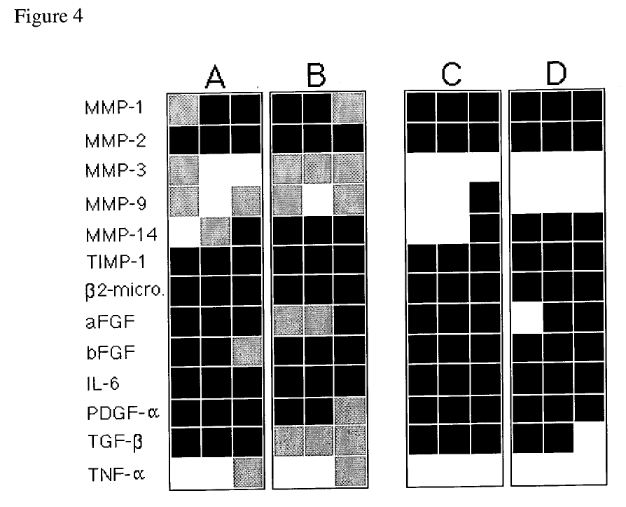

Modeling of a heterogeneous group of transcripts: In the second part of Example I, a heterogeneous set of genes including MMPs, tissue inhibitor of metalloproteinases, and various growth factors was modeled. Three groups of tissues were derived from chronic fibrosing patients, normal control, and Dupuytren's disease patients. We first predicted the mRNA level of one gene from the mRNA level of the other genes. When the mRNA level was assigned to 3 levels, the prediction by the least-square estimator gave the correct level in 26 (67%) out of 39 total cases (FIGS. 4A and 4B). Twelve cases were incorrect by a single expression level, and one case was off by two expression levels. Without a weight imposed for compensating variations among genes, the correct cases were reduced to 22 (56%). When the expression was assigned to 2 levels, the maximum rate of successful prediction increased to 34 cases (87%) (FIGS. 4C and 4D). Without any weighting factor, the correct prediction was limited to 28 cases (72%).

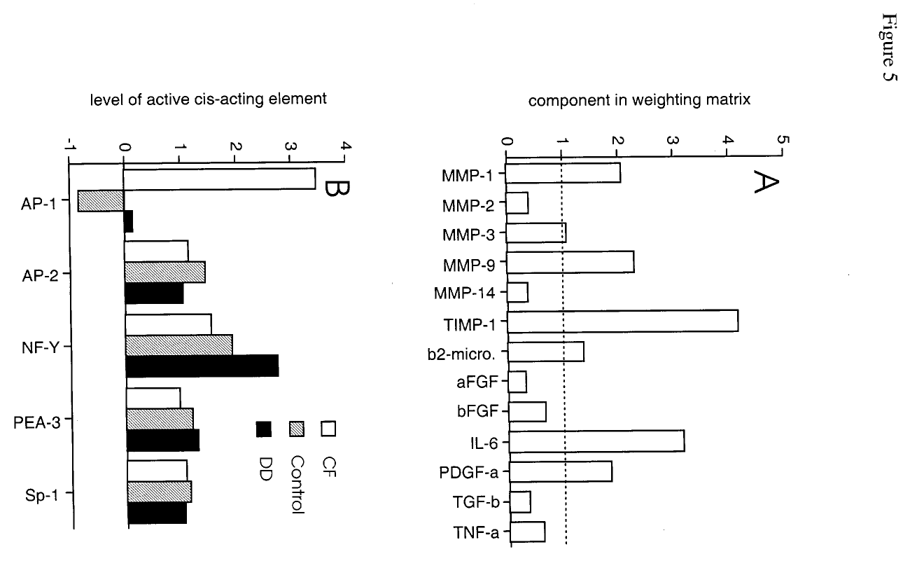

Variations among genes: Weighting factors were introduced to evaluate variations among genes. Since an element in the weighting matrix was assigned inversely proportional to the mean-square error (see formulation above), each element should serve as a fitness indicator of each gene. We have shown that performance of the least-square modeler was enhanced by the weighting matrix. A large element assigned to the genes such as TIMP-1, IL-6, MMP-9, and MMP-1 indicated that their measured mRNA levels fit well with the linear estimation model (FIG. 5A). A poor fitting of MMP-2, MMP-14, aFGF and TGF-β, on the other hand, was suggested by a low value. We excluded four genes such as IL-1α, IL-1β, TNF-β, and PDGF-β because of poor fitting.

Estimation of active cis-acting elements: The last step was to estimate a level of active cis-acting elements for three tissue groups derived from chronic fibrosing patients, control, and Dupuytren's disease patients (FIG. 5B). In chronic fibrosing patients, the active level of AP1 was significantly higher than the other two groups, while the estimated level of NF-Y was highest in Dupuytren's disease patients. Five sensitivity values (eigenvalues) corresponding to the selected cis-acting elements were 9.7, 2.1, 0.8, 0.005, and 0.003. The eigenvector (a combination of cis-acting elements) corresponding to the largest eigenvalue was (0.03, −0.30, −0.03, 0.65, 0.70)^{T} in a 5-dim space of AP1, AP2, NFY, PEA3, and Sp1. A large positive value for PEA3 and Sp1 suggested a stimulatory role and a large negative value for AP2 indicated an inhibitory role.

### Discussion

In an attempt to establish a systematic model for eukaryotic transcription activities, a promoter-based estimation algorithm was developed and a sensitivity analysis for the selected cis-acting regulatory elements was conducted. The described mathematical formulation allowed us to highlight the merits and limitations of linear approximation in analyzing complex eukaryotic transcriptional regulation.

Two merits of the described promoter-based estimation analysis are the capability of modeling and predicting mRNA levels and the unique sensitivity analysis for active cis-acting elements. One major difference between the current work and other linear regression models is a system's formulation (10). In our formulation a direct mRNA level rather than a logarithm of an expression ratio was used as a measurement variable, and an activation level of cis-acting elements was defined as a state variable. This formulation allowed us to estimate the state variables (cellular states) and to model and predict the measurement variables (mRNA levels) from the promoter matrix and the associated matrices. Our least-square modeler can accommodate, if necessary, supplementary data in the form of a priori information or weighting factors, and it can be extended into a dynamical model without altering the definition of state and measurement variables. In this study, 5-7 cis-acting elements were chosen from 500-bp promoters (part I of Example I) and 800-bp promoters (part II of Example 1). A careful determination of promoter length and cis-acting elements seems to further improve performance of the described least-square linear estimator. Although a model with 7 cis-acting elements was presented in part 1 of Example 1, the combination of 5 elements such as AP1, AP2, NFY, PEA3, and Sp1 gave the minimum model error for the 500-bp upstream regulatory sequences and different combinations of cis-acting elements were better for other regulatory regions (data not shown).

The sensitivity analysis provided a good measure for the combinatorial effects of cis-acting elements. A set of combinations of cis-acting elements, eigenvectors, represent independent (orthogonal) combinations in a space of cis-acting elements, and associated sensitivity values (eigenvalues) indicate the effectiveness of particular combinations of cis-acting elements in altering mRNA expression. The primary eigenvector corresponding to the largest eigenvalue indicates the most effective combination of cis-acting elements to regulate a mean-square sum of mRNA levels. For instance, the positive value in the primary eigenvector for AP1, PEA3, and Sp1 in two examples suggested a stimulatory effect of transcription factors such as c-fos, c-jun, and ets-1. On the other hand, AP2 had a negative value in both examples, suggesting an inhibitory effect. Although the above interpretation of AP1, PEA3, Sp1, and AP2 appears consistent with several lines of biochemical observations, a role of these elements depends on tissue samples, individual genes, and growth conditions (16-18). The linear PROBE model should be able to predict the role of cis-acting elements specific to individual tissues or genes.

The model in the PROBE analysis provides an approximation of eukaryotic regulatory networks. Genes that are regulated on a post-transcriptional level such as IL-1α and IL-1β did not fit the model (19). When an expression level was randomly assigned in the Monte Carlo simulation, the predicted mRNA level also became nearly random. Therefore, the linear model represents, at least in part, complex transcriptional machinery related inflammation and degeneration of skeletal tissues. Our analytical approach is justified for the following reasons. First, cis-acting elements are indispensable in transcription activities and the 5′-end regulatory promoter focused in this analysis represents a core region besides other regulatory regions located in 3′-ends or introns (20). Second, eukaryotic transcriptional activities are controlled by a combination of multiple cis-acting elements and a weighed sum of the number of cis-acting elements appears as a simplified representation of their contribution. Third, transcriptional assays such as a reporter gene assay and an electrophoretic mobility shift assay are able to simulate the functional significance of cis-acting elements using shorter DNA fragments in 20-500 bp than complete gonomic DNA sequences (21).

The PROBE analysis for modeling and analyzing transcription activities provided in Example I offers a computational tool for life scientists and biomedical engineers to integrate experimental expression data with available genome information. It was a unique application of a linear estimation theory popularly used in navigating spacecraft or processing electric signals (25). In this study we started with the smallest number of essential components, since an elegant model can often have greater intrinsic value than an accurate one overloaded with detail (26). We did not follow a commonly accepted scheme of modeling that requires a number of parameters related to binding affinity and stability of trans-acting regulatory elements (27). Although the linear model described here is an approximation of complex eukaryotic regulation, a simple, but general mathematical framework provided logical insights in a combinatorial role of cis-acting elements.

### EXAMPLE II

### Promoter Competition Assay For Analyzing Gene Regulation in Joint Tissue Engineering

A “promoter competition assay,” for examining the role of cis-acting DNA elements in tissue cultures is provided in the present example. Recent advances in tissue engineering permit the culture of a variety of cells. Many tissues are engineered, however, without an appropriate understanding of molecular machinery that regulates gene expression and cellular growth. For elucidating the role of cis-acting regulatory elements in cellular differentiation and growth, we have developed the promoter competition assay. This assay uses a transient transfer into cells of double-stranded DNA fragments consisting of cis-acting regulatory elements. The transferred DNA fragments act as a competitor and titrate the function of their genomic counterparts. Using synovial cells derived from a rheumatoid arthritis patient, we examined a role of NF-κB binding sites in the regulation of matrix metalloproteinase genes. The results provide a proof of concept for the method of the present invention as altered mRNA expressions and a retarded cellular growth were observed.

The availability of a complete set of human genome sequences provides an exciting, challenging opportunity for tissue engineers (1, 2). For engineering a specific type of tissue such as cartilage (29), genomic DNA sequences are a rich resource useful in elucidating molecular mechanisms underlying tissue growth and differentiation. In testing a role of regulatory DNA sequences, however, a conventional assay such as a reporter gene assay or an electrophoretic mobility shift assay is not well suited to deal with a large volume of databases in the post human genome project era (30). There is an escalating need for a new assay that provides an efficient functional test of regulatory DNA elements.

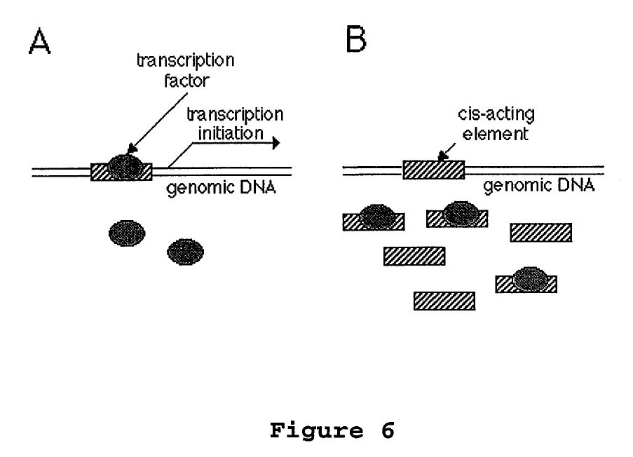

In order to efficiently test for the role of genomic promoter sequences, we developed a new biochemical assay referred to herein as “promoter competitor assay.” In this assay DNA fragments consisting of a specific cis-acting element are transiently transferred into cultured cells. Alterations in the mRNA level of genes of interest are monitored by reverse transcription and PCR (FIG. 6). Since exogenous DNA fragments act as a competitor of genomic cis-acting elements, reduction in specific gene transcripts in the assay suggests that the transferred cis-acting elements mimic the binding capacity of endogenous cis-acting elements. Preparation of DNA fragments is straightforward compared to preparation of plasmid vectors or antibodies, and therefore a scaled-up systematic assay for the role of putative cis-acting elements can be readily performed.

In this study, we examined the promoter competition assay by using synovial cells derived from a rheumatoid arthritis patient (30). Rheumatoid arthritis is a chronic joint disease caused by complex interactions with an immune system. Inflammation and degradation of soft joint tissue are major symptoms (8, 31). It is imperative for tissue engineers to identify gene regulation mechanisms that are specific to rheumatic auto-inflammatory responses. In that way it might be possible to generate tissue resistant to inflammation or degradation. Suppressing the expression of proteolytic enzymes such as matrix metalloproteinases (MMPs) is one approach to alleviate rheumatic symptoms (11, 12, 15). Focusing on a role of NF-κB in the transcriptional regulation of MMPs, we demonstrate here that mRNA levels of MMP-1 and MMP-13 in synovial cells are diminished in the promoter competition assay. NF-κB is shown to stimulate inflammatory responses in rheumatic joints (32). Since synoviocytes are under shear stress in joints, the cells for this study were grown under 0-10 dyn/cm² shear stress. The results provided a proof of concept, supporting the use for the promoter competition assay in identifying roles specific cis-acting elements play in gene regulation. Elucidating such roles is an important challenge for tissue engineering.

The following materials and methods are provided to facilitate the practice of Example II

Cell Culture

A human synovial cell line (MH7A, Riken Cell Bank, Japan) derived from the intra-articular soft tissue of the knee joint of a rheumatoid arthritis patient, was used in this study (30). MH7A cells are fibroblast-like synoviocytes immortalized by transfection with SV40 T antigen. The cells were spread on a glass slide coated with type I collagen and grown in the RPMI 1640 medium supplemented with 10% fetal calf serum and antibiotics at 37° C. We used cells at ˜80% confluency 3-4 days after fresh spreading.

Promoter Competition Assay

Cells were incubated with double-stranded DNA fragments consisting of a specific cis-regulatory element. The mRNA level of target genes was determined by reverse transcription and polymerase chain reactions (RT-PCR). In the current study, we used two 18-bp double-stranded DNA fragments to test a role of NF-κB: 5′-TGCAGGGGATYCCCGACT-3′; (SEQ ID NO: 1) (including NF-κB binding site) and 5′-TGCAGACTCATGTAGCGT-3′; (SEQ ID NO: 2) (random sequence) as a control. Cells were transiently incubated with 0.05-5 μM DNA fragments that acted as a competitor of the corresponding genomic cis-acting elements. DNA fragments with and without phosphorothioate modification (Ana-Gen Technologies, Inc.) were used.

Mechanical Shear

Since joint cells in vivo are under shear stress, mechanical shear stress was applied to MH7A cells using a Streamer Gold flow device (Flexcell International Corp.). The device generates a Poiseuille flow within a pair of parallel plates separated by 500 μm and induces uniform fluid shear. Cells were grown in a monolayer in the medium consisting of 10% fetal calf serum. Shear stress at 1, 2, 5, and 10 dyn/cm² was applied for 1 hour.

Reverse Transcription and Polymerase Chain Reaction (RT-PCR)

Total RNA was isolated using an RNeasy mini kit (Qiagen, CA), and the isolated RNA was reverse-transcribed by MMLV reverse transcriptase using random primers. The cDNAs corresponding to MMP-1, MMP-8, MMP-13, and glyceraldehyde-3-phosphate dehydrogenase (GAPDH, control) were amplified by PCR (GeneAmp PCR System 2400, Perkin Elmer). The PCR primers are listed in Table 2 (18). The PCR included 25 cycles at 94° C. for denaturation (1 min), 54-62° C. for annealing (30 sec), and 72° C. for extension (30 sec). All experiments were performed three times to demonstrate reproducibility.

TABLE 2
PCR primers used Example II

| Gene | sense primer | Antisense primer | CDNA size (bp) |
|---|---|---|---|
| MMP-1 | CACAGCTTTCCTCCACTGCTGCTGC | GGCATGGTCCACATCTGCTCTTGGC | 396 |
| MMP-8 | TAAAGACAGGTACTTCTGGAGAAGG | GCTTCAGCGATATCTACAGTTAAGC | 514 |
| 6 | | | |
| MMP-13 | TGGTGGTGATGAAGATGATTTGTCT | AGTTACATCGGACCAAACTTTGAAG | 376 |
| 6 | | | |
| GAPDH | CCACCCATGGCAAATTCCATGGCA | TCTAGACGGCAGGTCAGGTCCACC | 600 |

Cellular Growth Analysis

In order to examine effects of incubated DNA fragments on cellular growth in the promoter competition assay, cell number was monitored after transferring DNA fragments. The synovial cells were incubated with 5 μM of DNA fragments for 1 hour on day #1 and day #2, and harvested for determining the growth rate (cell number) on day #3. The mean number of cells and the standard deviation were determined for the normal control, the control incubated with random DNA sequences, and the experimental cells incubated with DNA fragments consisting of NF-κB cis-acting elements.

### Results

Response to Mechanical Shear

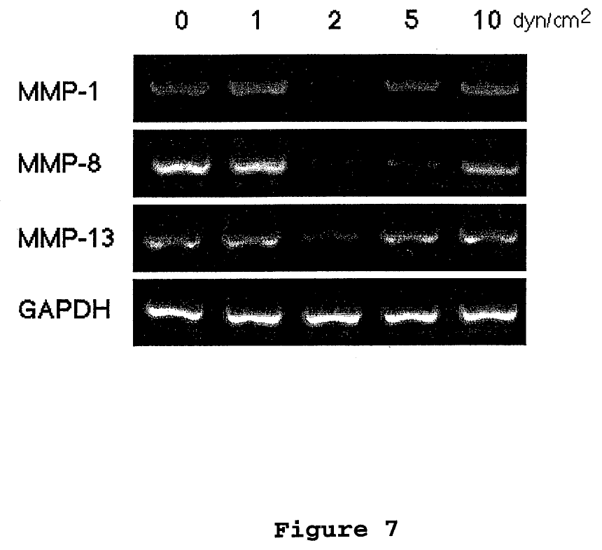

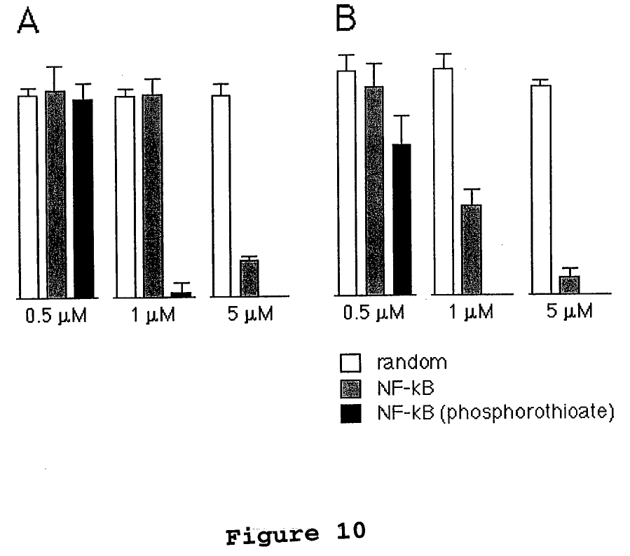

Prior to employing the promoter competition assay we first determined basal mRNA levels for three MMPs. Since synovial cells in vivo are under mechanical stress, we applied flow shear to cells for 1 hour at varying intensities of 0-10 dyn/cm². RT-PCR revealed that under gentle shear at 2 dyn/cm² the mRNA level of MMP-1, MMP-8, and MMP-13 was decreased (FIG. 7). However, under high shear at 10 dyn/cm², the mRNA level of three MMPs was elevated up to the basal control level (FIG. 10). The results support the previous finding that the level of MMP mRNA is shear stress dependent. The optimal stress to minimize the mRNA expression of MMPs exists at a few dyn/cm² (18).

Promoter Competition Assay Using NF-κB Cis-Acting Elements

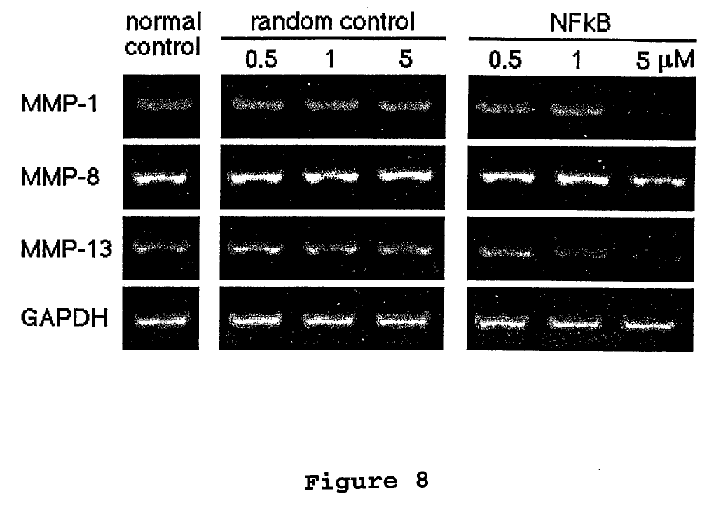

In synovial tissue derived from a rheumatoid arthritis patient, suppression of proteolytic enzymes such as MMPs is thought to contribute to preventing tissue degradation. In employing the promoter competition assay, we focused on the cis-acting element consisting of the NF-κB binding sites. Synovial cells were incubated with 0.05-5 μM double-stranded DNA fragments for 1 hour. We used two different DNA fragments, one consisting of NF-κB binding sites and the other containing random DNA sequences. The cells were harvested immediately after incubation with exogenous DNA fragments. RT-PCR was then conducted to determine the level of NMP mRNAs (FIG. 8). When cells were incubated with 5 μM of DNA fragments, the mRNA level of MMP-1 and MMP-13 was reproducibly reduced. However, the incubation with the same amount of random DNA fragments did not alter MMP expression. The expression of MMP-8 was not affected in the assay with any of the DNA fragments.

Promoter Competition Assay Under Mechanical Shear

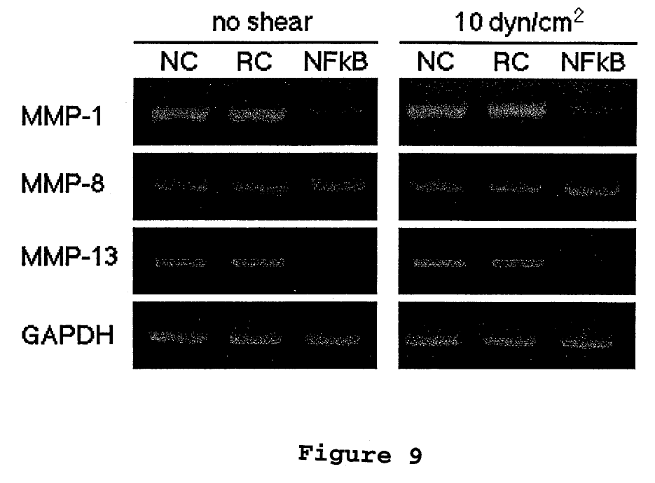

We next employed the promoter competition assay to examine the cells under mechanical shear, since joint tissue in vivo is under constant shear. MH7A cells were grown for 1 hour under 0 dyn/cm² (control) or 10 dyn/cm² shear after 1-hr incubation with 5 μM of double-stranded DNA fragments. The RT-PCR results showed that the DNA fragments consisting of NF-κB binding sites reduced the mRNA expression of MMP-1 and MMP-13 with and without mechanical shear (FIG. 9). The incubation with the random DNA sequences did not alter any mRNA expression (FIG. 9). The expression of MMP-8 was unaltered in a promoter competition assay with either DNA elements (FIG. 9).

In antisense DNA applications modified DNA oligonucleotides are often used to increase efficiency of a DNA transfer and to enhance stability of transferred molecules. We used the DNA fragments with and without phosphorothioate modification and compared effectiveness in suppression of MMP transcripts. The DNA fragments with phosphorothioate modification were more effective in reducing the mRNA expression of MMP-1 and MMP-13 than the fragments without modification (FIG. 10). For instance, the modified fragments at 1 μM nearly abolished the expression of MMP-1 mRNA, but the unmodified counterpart at 1 μM did not alter it expression. The expression of MMP-8 mRNA was not altered by either DNA construct.

Effects of NF-κB Cis-Acting Elements On Cellular Growth

Realizing that exogeneous NF-κB cis-acting elements induce a reduction in the mRNA expression of MMP-1 and MMP13, we lastly examined effects of this competitor on cellular growth. During a 3-day culturing period, cells were exposed twice to 5 μM DNA fragments for 1 hour in day #1 and day #2. The number of cells was determined as 13.5±2.8 (mean ± standard deviation in an arbitrary unit; normal control), 11.9±2.2 (control with random DNA fragments), and 9.3±1.5 (experimental with NF-κB DNA fragments). The reduction in the number of cells in the experimental was statistically significant compared to the normal control (p<0.0001 in t-test). No statistical significance was detected between the normal control and the control with random DNA fragments (p=0.09). Since rheumatic cells proliferate at a higher growth rate than normal synovial cells, a retarded growth rate by NF-κB elements can be considered a favorable phenotype.

The described promoter competition assay of the present invention provides a new technique for testing the functional significance of cis-acting elements in cultured cells. Using synovial cells derived from a rheumatoid arthritis patient as a model system, we tested a role of NF-κB binding sites in the mRNA expression of three MMPs. The results showed that (i) the expression of three MMPs was sensitive to flow shear and the response was stress-intensity dependent; (ii) the mRNA expression of MMP-1 and MMP-13, but not MMP-8, was suppressed in the promoter competition assay using NF-κB cis-acting elements; and (iii) cellular growth was retarded by the competitive NF-κB assay. Since the assay using random DNA fragments did not affect the MMP expression or cellular growth, the results in this study support a stimulatory role of NF-κB binding sites for the expression of MMP-1 and MMP-13 as well as cellular growth.

In rheumatoid arthritis, NF-κB plays an essential role in transcriptional activation induced by TNF and IL-1 (32). The promoter competition assay revealed a differential role of NF-κB binding sites among three MMP genes tested here. In the 5′-end regulatory sequences of many MMPs, putative NF-κB binding sites have been identified. There are 1, 2, and 1 site in an 800-bp promoter of MMP-1, MMP-8, and MMP-13, respectively. In the current assay using NF-κB cis-acting elements, the mRNA level of MMP-1 and MMP-13 was reduced but the level of MMP-8 mRNA was unchanged. Since the most MMP genes are co-regulated by AP-1, AP-2, and PEA-3 sites, multiple cis-acting elements besides NF-κB appear to regulate the expression of MMPs (13, 22, 33). In the separate experiments the DNA fragments consisting of AP-1 site (TGACGTNTGASTCAGCATGC) partially reduced the mRNA expression of MMP-1 but the fragments containing AP-2 site (TGCAMKCCCSCNGGCGGACT) did not alter the expression (data not shown). The results suggest that the combinatorial effect of multiple cis-acting elements can be tested in this assay.

Although the current study provides “proof of concept,” the efficiency of transferring and stability of DNA fragments may be further enhanced. In this study, 0.05-5 μM DNA fragments without any modification was primarily used and 1-hour incubation with 5 μM DNA fragments yielded significant effects on gene regulation and cellular growth. The higher concentration of 10 μM did not differ from the results obtained at 5 μM. Phosphorothioate-modified nucleotides were a more potent suppressor of the mRNA expression of MMP-1 and MMP-13, and other DNA modifications used for antisense DNA may further enhance effectiveness of transferred DNA fragments (34, 35).

Like the impact of modern molecular biology on research projects in embryology, elucidation of a complex regulatory network is indispensable for engineering tissue differentiation and growth. This assay facilitates the identification of the role of cis-acting elements with reference to a complete set of human genomic sequences. The described assay is suited for a systematic examination of multiple cis-acting elements and a micro-fabricated cell plate can be designed. In further applications, cells can be cultured on a micro DNA array that provides a source for cis-acting elements to be tested (36).

Tissue engineering strategies are multifaceted, and are comprised of several components and features. Included is the preparation and culture of appropriate starter cells for differentiation; design of functional matrices for physically supporting those cells while they multiply and specialize; surgical insertion methods; and the design and administration of drugs/growth factors to regulate the transition of the starter cells from a dissociated and/or cultured state to an integrated/interconnected functional cell mass (37). Substantial progress has been made for several aspects of those strategies. The most refractory aspect will likely continue to be in the area of regulating gene expression, both qualitatively and quantitatively, in cells that comprise the engineered tissue.

The promoter competition assay described herein represents a model system for the design of future experiments. It should be possible, for example, to extend the principle of this assay for elucidating other cis-acting regulatory components, such as silencers and enhancers (38, 39). As well, it should be possible to use this same principle for assessing the extent to which a cis-acting nucleotide sequence is involved in the coordinate regulation of other—perhaps presently unrecognized—transcription events. Using either differential hybridization or gene microarrays for elucidating complex gene expression patterns, accurate profiles of regulation of gene expression in cells targeted for tissue engineering can be achieved. Once those profiles are available, rational drug design and educated searches for appropriate growth factors for enhancing proper differentiation in starter cells for tissue engineering can proceed with higher expectations for success (40).

Human genome sequence data will provide the driving force for patterning large-scale screens in which various cell types which are candidates for tissue engineering are cultured directly on micro DNA array plates which contain putative cis-acting sequences. With appropriate gene expression markers the regulatory circuitry that controls phenotypic expression will be elucidated. This information provides the basis for more accurate choices of cell type and/or sub-populations of heterogeneous tissues for replacement therapy.

### EXAMPLE III

### Model Based Analysis Of cDNA Microarray Data On Interleukin-1 Responses in Human Chondrocytes

A novel model-based analysis for identification and evaluation of transcription factor (TF) binding motifs in eukaryotic gene regulation is provided in the present example. Using the responses to interleukin 1 (IL1) in human chondrocytes as a model system, the functional TF binding motifs in the responses to IL1 were identified through mathematical formulation, and the model-based prediction was examined by a biochemical assay. In building a mathematical model, we first selected 45 genes responsive to IL1 from the cDNA microarray-generated data. For each IL1-responsive gene, frequencies of any 5-bp oligonucleotides in the upstream regulatory DNA region were determined. The mathematical model was then built to establish a quantitative relationship between the altered mRNA level of the IL1-responsive genes and the level of significance of TF binding motifs. The putative TF binding motifs, predicted by the model, included a consensus sequence for a GC box and an NFκB binding site as well as novel DNA elements such as CAGGC and CCGCC. By transferring the DNA fragments consisting of CAGGC as a competitor to the endogenous counterpart, the model-based prediction of its stimulatory role was validated. The genome-wide model-based approach described herein has its advantage in extracting biologically meaningful information from a large volume of expression data and genomic DNA sequences. The results support that the described mathematical and biochemical approach facilitate the identification and evaluation of critical TF binding motifs in complex transcriptional processes.

In order to interpret a large volume of microarray-generated expression data in light of genomic DNA sequences and to extract biologically meaningful information critical in gene regulation, a mathematical and computational model-based approach has an advantage (44). Pioneering works were conducted mainly for yeast gene regulation, and known and unknown regulatory DNA elements were analyzed using a linear regression model (10).

In this example, the mathematical model described in Example I for an analysis of the mRNA level of a family of matrix metalloproteinase genes in human synoviocytes (11, 50), was extended to a genome-wide model. In the original model, a quantitative relationship between the observed mRNA level of matrix metalloproteinase genes and frequencies of the selected TF binding motifs on the upstream regulatory DNA sequences was established. Using unique state-variable representation, a cellular state for each tissue sample was defined as a level of significance for each TF binding motif. Although the model was useful to distinguish the expression pattern with rheumatoid arthritis from the pattern without it, the PCR-based expression dataset was relatively small and only known TF binding motifs such as AP1, AP2, PEA3, and Sp1 were considered.

In the present example, we used the cDNA microarray data for the responses of human chondrocytes to interleukin 1 (IL1) as a model system (54). Like TNF-α, IL1 is a proinflammatory cytokine upregulated in joint tissue of patients with rheumatoid arthritis (42). Since it stimulates inflammatory responses and tissue degeneration, an understanding of the IL1-mediated tissue degradation is critically important. In the current model, we started with 5-bp random DNA sequences (512 in total) as potential TF binding motifs and searched for a meaningful combination of putative TF binding motifs. In order to evaluate efficiently a large number of combinations, we developed an eigengene-eigenTF analysis. In this analysis, the observed mRNA pattern for the IL1-responsive genes was described as a linear combination of the eigengene vectors and the corresponding linear combination of the eigenTF vectors was used to evaluate functional significance of 512 random DNA sequences.

In order to validate any theoretical model, model-based prediction should be examined not only by statistics but also by a biochemical assay. Here, the predicted role of the critical putative TF binding motifs was evaluated by a promoter competition assay described in Example II. In this assay the DNA fragments consisting of a specific TF binding motif were transferred into chondrocyte cells, and any alteration in mRNA expression by the DNA transfer was monitored in order to estimate functional significance of the transferred TF binding motifs. The results supported a potential use of the described approach in identification and evaluation of critical TF binding motifs.

Materials and Methods to Facilitate the Practice of Example III

Using the responses to IL1 in human chondrocytes as a model system, we developed and applied the integrated mathematical, biochemical procedure to identify and evaluate critical TF binding motifs. The analysis was composed of the four major steps including “identification of IL1-responsive genes,” “definition of promoter matrix,” “selection of putative TF binding motifs,” and “evaluation of putative TF binding motifs.”

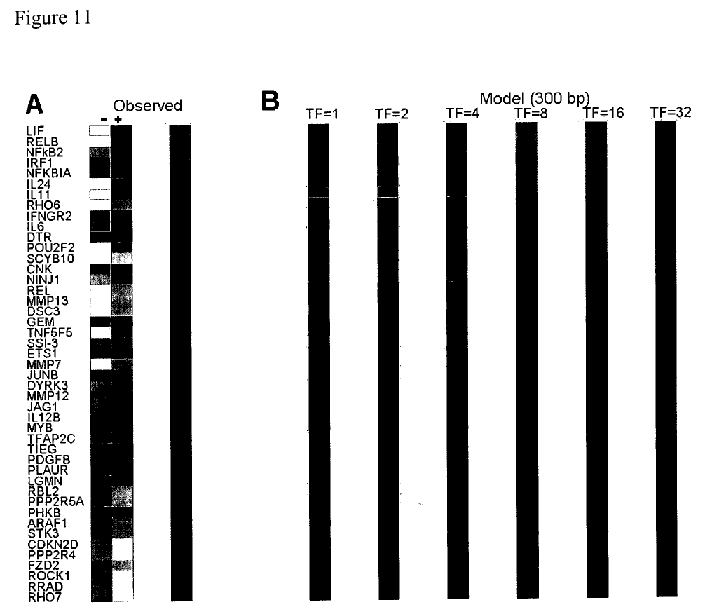

Identification of IL1-responsive genes: The expression data for the IL1-responsive genes in human chondrocytes were obtained from the tables published in (54). We focused on 45 IL1-responsive genes (FIG. 11). Their mRNA expression level was either increased or decreased by twofold or more in the presence of 10 ng/ml IL1β, and the transcription initiation site was identifiable in GenBank sequences or by prediction using a PEG program (55, 43).

Definition of promoter matrix H: In mathematical formulation of the responses to IL1, we defined a (n×m) promoter matrix H (n=number of the IL1-responsive genes; and m=total number of putative TF binding motifs) in the following three steps. First, the upstream DNA sequences of the 45 IL1-responsive genes (n=45) were retrieved from human genome DNA sequences. Second, frequencies of 5-bp random DNA sequences (512 in total without considering polarity; m=512) was counted as potential TF binding motifs in the 5′-end flanking regulatory region. The (i, j) component of the promoter matrix H, hᵢⱼ, stored the number of appearance of the j-th random DNA sequence on the upstream regulatory region of the i-th IL1-responsive gene. Third, in order to characterize a contribution of each DNA fragment to the responses to IL1, the promoter matrix H was factorized using singular value decomposition:

H=UΛV^{T} (1)

where U=an eigengene matrix u₁, u₂, . . . , uₙ), Λ=a diagonal matrix whose diagonal components were composed of the eigen values such as λ₁, λ₂, ,,, λᵣ, V=an eigenTF matrix (v₁, v₂, . . . , vₘ), and r=rank of the matrix H.

Selection of putative TF binding motifs: Selection of putative TF binding motifs was done in three steps through the eigengene-eigenTF analysis. First, we built a quantitative relationship between the altered mRNA levels and the change in cellular states in response to IL1. Using the promoter matrix H, the mRNA level of the IL1-responsive genes was modeled:

z=Hx (2)

where z=an observation vector representing the altered mRNA level by IL1, and x=a cellular state vector indicating the shift in a level of significance of the TF binding motifs. We defined z as a logarithmic ratio of the mRNA level with IL1 to the mRNA level without IL1. Second, a linear combination of the eigengene vectors was formed:

□=Σkᵢλᵢuᵢ (3)

for i=1 to r, where kᵢ was a weighting factor for the i-th eigengene. Third, functional TF binding motifs were selected in the order of a weighting factor imposed to each DNA element in a linear combination of the eigenTF vectors in a form, Σkᵢvᵢ(i=1 to r).

Evaluation of putative TF binding motifs: In order to evaluate statistical significance of the results derived from the model-based analysis, we performed a Monte Carlo simulation. In the simulation, the observed mRNA level was scrambled and mean modeling error was computed from 10,000 trials. We also conducted a promoter competition assay and evaluated biochemically the role of the two putative TF binding motifs, CAGGC and CCGCC (See Example II). These two sequences were identified through the mathematical model as the stimulatory and inhibitory elements in the IL1 responses, respectively. Human chondrocytes (C-28/I2 cell line, Loeser et al. 2000) were grown in the presence of 5 μM of the 15-bp double-stranded DNA fragments consisting of 5′-ATCAGCAGGCATACG-3′ or 5′-ACAATCCGCCGTTTA-3′). The underlined sequence, used to extend the length of DNA fragments, was chosen from the 5-bp DNA sequences that did not provide any significant role in the mathematical model. Total RNA was isolated, and RT-PCR was conducted using the primers for the three IL1-responsive genes (LIF, NFκB, and IRF1) listed in Table 3.

TABLE 3
PCR primers

| | Sense | antisense | |
|---|---|---|---|
| Gene | Primer | primer | CDNA size (bp) |
| LIF | 5′-GCCAAGCTGGTGGAGCTGTA-3′ | 5′-ACGGCGATGATCTGCTTATA-5′ | 293 |
| NFκB | 5′-TCTGAGTATAGTGCGGCTGC-3′ | 5′-AACACTGTTACAGGCCGCTC-3′ | 360 |
| IRF1 | 5′-GCAGCTCAGCTGTGCGAGTG-3′ | 5′-CTGCCACTCCGACTGCTCCA-3′ | 438 |

### Results

The mRNA expression of the IL1-responsive genes was modeled using the 300-bp upstream regulatory region. Among the 45 IL1-responsive genes analyzed in this report 33 genes was upregulated in response to IL1, while 11 genes were downregulated. Using the mathematical model described in Materials and Methods, the observed alteration in mRNA levels was modeled using the 300-bp upstream regulatory DNA region (FIG. 11). We employed the eigengene-eigenTF analysis and selected 1-32 members of 5-bp DNA sequences as putative TF binding motifs. Modeling error, defined as the mean-square sum of the difference between the observed and the modeled mRNA levels, decreased monotonically as the number of the selected DNA elements from 1 to 32. Hereafter, we focused on analyzing the model with 8 members of 5-bp putative TF binding motifs.

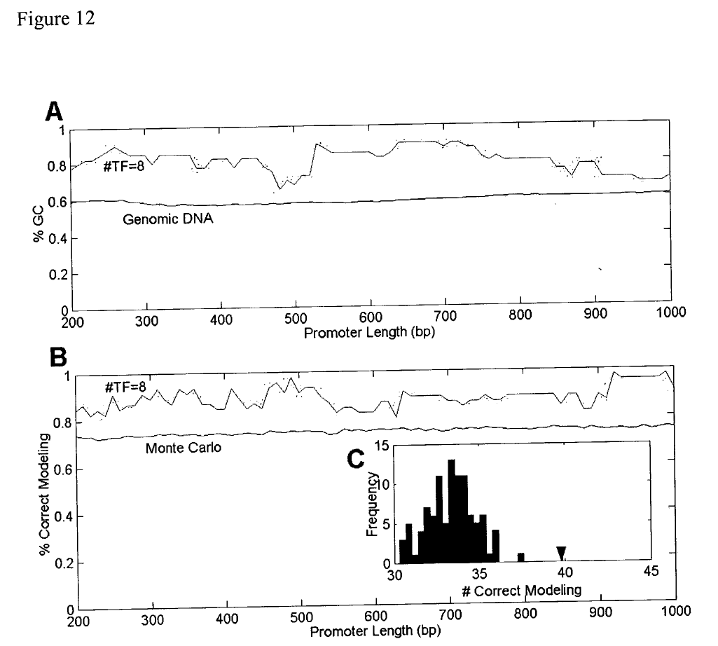

With <1,000-bp upstream regulatory DNA sequences, the responses to IL1 were modeled correctly at ˜90%. For the upstream regulatory DNA region 200-1,000 bp in length, we determined the combination of 8 putative TF binding motifs with a minimum modeling error. The model was able to predict significantly better than the Monte Carlo simulation with the scrambled expression data as to whether the effects of IL1 would be up- or downregulation for individual genes (FIG. 12). Interestingly, the upstream DNA sequences of the IL1-responsive genes had ˜60% of GC contents and the selected putative TF binding motifs had 80% (FIG. 12).

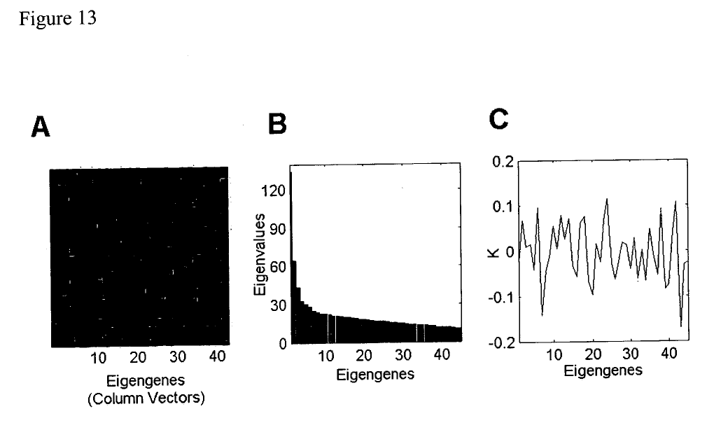

The mRNA level of the 45 IL1-responsive genes was modeled as a linear combination of the eigengene vectors. Using singular value decomposition, the promoter matrix H, built from the 300-bp upstream regulatory DNA sequences, was factorized into three matrices such as U, Λ, and V. The eigengene matrix, U, consisted of the 45 eigengene vectors, and the observed mRNA expression for the 45 IL1-responsive genes was modeled as a linear combination of the eigengene vectors (FIG. 13). The weighting factors for the linear combination were used to select the putative TF binding motifs in the following procedure (FIG. 13).

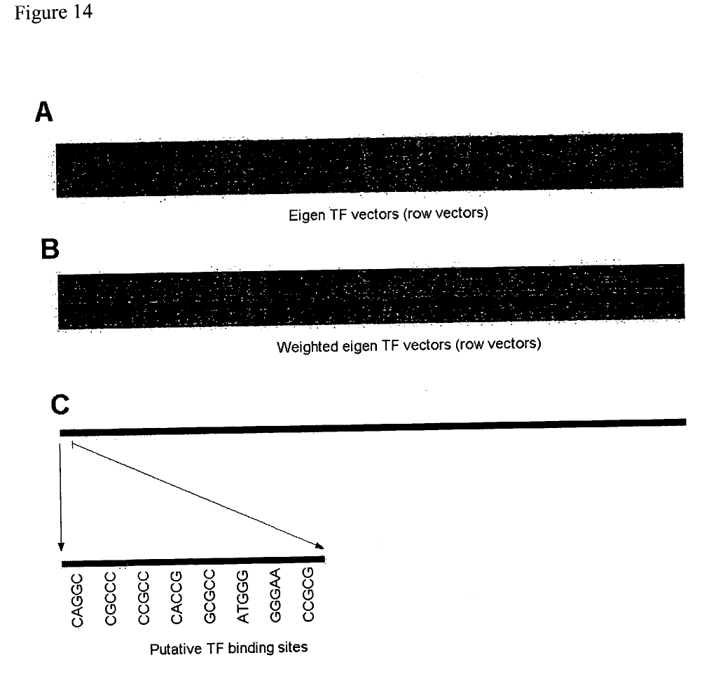

Model-based identification of putative TF binding motifs included a consensus sequence for a GC box and an NFκB binding site. Using the weighting factors illustrated in FIG. 3 and the eigenTF matrix, V, we selected 8 putative TF binding motifs in the responses to IL1 from 5-bp DNA sequences (512 in total). Based on the linear combination of the 45 eigenTF vectors using the procedure described in Materials and Methods, a contribution of each 5-bp DNA sequences to the responses to IL1 was evaluated (FIG. 14). The best 8 DNA sequences selected as putative TF binding motifs were CAGGC, CGCCC, CCGCC, CACCG, GCGCC, ATGGG, GGGAA, and CCGCG in the order of fitting to the model. Interestingly, the second and the seventh DNA sequences are identical to the consensus sequence for GC box and NFκB binding site.

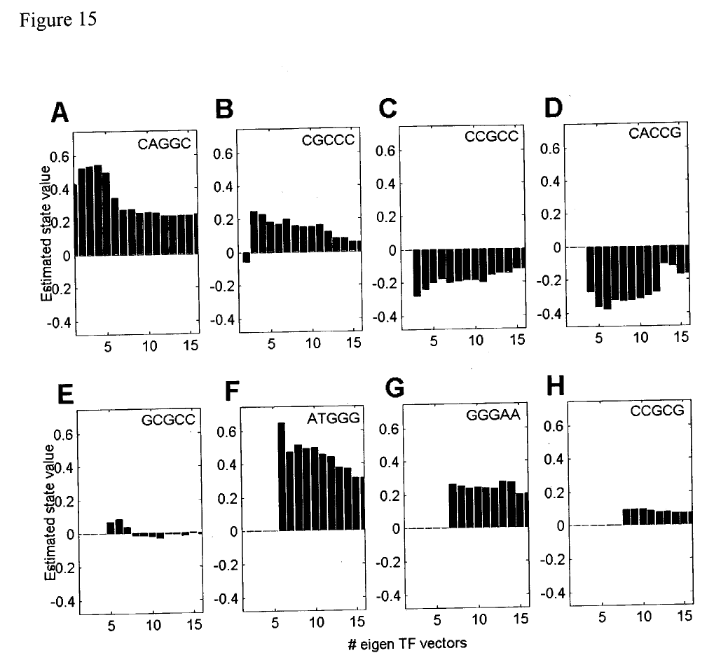

The model predicted a stimulatory role of 5 elements and an inhibitory role of 2 elements in the responses to WI. The cellular state, represented by x, was an indicator of the stimulatory or inhibitory role of TF binding motifs in response to IL1. Using the promoter matrix with the 300-bp upstream regulatory DNA region and a least-square estimation procedure, we determined the cellular state for the putative TF binding motifs (FIG. 14). The positive value for the estimated state implies the stimulatory role, and the negative value indicates the inhibitory role upon IL1 stimulation. Out of the 8 selected DNA fragments for the model with 1-16 TF binding motifs, 5 elements (CAGGC, CGCCC, ATGGG, GGGAA, and CCGCG) were consistently stimulatory, and two elements (CCGCC, and CACCG) were inhibitory. See FIG. 15.

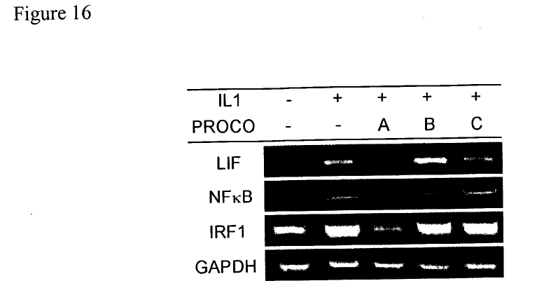

The promoter competition assay validated model-based prediction of the stimulatory role of CAGGC in response to IL1. Using chondrocytes incubated with the putative TF binding motifs, we determined the mRNA level of three IL1-responsive genes (LIF, NFκB, and IRF1) and evaluated the predicted role of CAGGC and CGCCC (FIG. 16). The expression of these three genes was upregulated 15-fold or more by IL1 in the microarray experiments (54). The DNA fragments consisting of CAGGC suppressed the IL1-induced increase in the mRNA level, validating the predicted stimulatory role of the DNA fragments. Although the LIF mRNA level was slightly upregulated by the DNA fragments consisting of CGCCC, its predicted inhibitory role was not detectable in the expression of NFκB and IRF1. The IL1-induced mRNA elevation was not altered by the control DNA fragment, which was composed of DNA sequences insignificant to the IL responses.

Discussion

The novel model-based analysis of the response to IL1 in human chondrocytes, focusing on the 45 IL1-responsive genes identified in the cDNA microarray experiment is provided herein. The described approach successfully integrated mathematical formulation that identified the putative TF binding motifs, with the biochemical assay that evaluated the role of individual TF binding motifs. Starting from the 5-bp random DNA sequences (512 in total) as putative TF binding motifs, the eigengene-engenTF analysis determined a level of significance of individual DNA elements and assigned the critical stimulatory and inhibitory elements in the responses to IL1. The role of the selected TF binding motifs was examined by the promoter competition assay, and the DNA sequence of CAGGC was validated as the novel stimulatory element in the IL1 responses.

The eigengene-eigenTF analysis provided an efficient means to search for putative TF binding motifs in a framework of linear algebra. Unlike factorizing mRNA expression datasets in a matrix form (7), we applied singular value decomposition to the promoter matrix, H, and derived the eigengene matrix, U, and the eigenTF matrix, V. Using U and V, a linear combination of the observed mRNA pattern leaded to estimate the level of significance of individual TF binding motifs. Although the resultant combination of the selected TF binding motifs may not be globally optimal in terms of modeling error minimization, the procedure does not require evaluation of 1.1×10¹⁷ (₅₁₂C₈) combinations for selection of 8 DNA elements from 512 candidates. Thus, the described eigengene-eigenTF analysis is suited for evaluating combinatorial effects of many potential TF binding motifs.

The described analysis for the IL1-responsive genes pointed out the stimulatory role of GC box and NFκB. Unlike the previous models using the known TF binding motifs such as AP1, PEA3, and Sp1, we built the mathematical model in this report using all possible combinations of 5-bp DNA fragments and searched for the putative TF binding motifs. Out of the 8 selected sequences, three sequences were linked to the GC box and one sequence was a part of the consensus sequence of the NFκB binding site. The GC box is a relatively common promoter component like a TATA box, and the consensus sequence is 5′-GGGCGG-3′. NFκB is a pivotal transcription factor in chronic inflammatory diseases including rheumatoid arthritis, and its activation by proinflammatory cytokines such as IL1 and TNF-α is well studied (41, 46). These results support the utility of the described approach in identifying and evaluating critical TF binding motifs in the complex biological process.

Two important factors in the current model building are a starting set of potential TF binding motifs and selection of regulatory DNA regions. In this report, an initial set of 5-bp DNA fragments in the <1000-bp 5′-end flanking regulatory DNA region was analyzed. The results suggested functional significance of 5-bp DNA elements on ˜300 bp promoter sequences.

The current example describes the novel model-based approach in interpreting the IL1-responsive gene expression obtained from the cDNA microarray data. Without using any fitting parameters, the state-variable representation and the eigengene-engenTF analysis allowed us to identify key TF binding sites from a pool of 512 random DNA sequences in the IL1-responsive eukaryotic regulatory system. The promoter competition assay was able to validate the model-based prediction and to update the mathematical model by feedback from the assay results. The described approach is applicable to other biological processes, and we believe that it will contribute to extract biologically meaningful information on complex gene regulatory circuits.

### EXAMPLE IV

### Model Bases Analysis of Matrix Metalloproteinase Expression in Synovial Cells Grown Under Mechanical Shear

In the present example, the development of a model-based analysis for identification of the role of transcription factor (TF) binding motifs is provided. A nonlinear mathematical model was formulated to establish the quantitative relationship between the temporal expression profiles and the distribution of known TF binding motifs on regulatory DNA regions. In order to evaluate biological meaning of the nonlinear model, the role of TF binding motifs predicted by the model was examined by a promoter competition assay where specific TF binding motifs were inactivated by a transient transfer of the DNA fragments consisting of the TF binding motifs. Using the shear stress responses of a family of matrix metalloproteinases in human synovial cells as a model system, we showed that the nonlinear formulation more closely approximates the experimentally observed expression profile than the linear formulation. Also, the stimulatory and inhibitory role of TF binding motifs extracted from the model was validated by the competition assay. The results support that an integrated usage of the linear and nonlinear models and the biochemical evaluation assay enables the identification of critical regulatory DNA elements for tissue engineering.

In order to control growth and development of tissues in vitro and ex vivo, it is indispensable to identify critical regulatory DNA elements. Here, we describe a novel model-based gene expression tool that would predict and evaluate the cellular state of transcriptional regulation using regulatory DNA sequences of individual genes.

In order to develop the model-based analysis, we treated the transcriptional machinery as a nonlinear control system and defined the state of the system using a set of time-varying variables. In the present model, the input to the system was fluid-driven shear stress and the output was a set of mRNA expression levels. The output was represented by the cellular state values that were defined from the activation level of known transcription factor (TF) binding motifs such as AP1, AP2, NFκB, etc. Previously, we developed the linear mathematical model that established the relationship between the state variables and the output measurement. See Examples I and III. The linear model allowed interpretation of mRNA expression profiles from the distribution of various TF binding motifs on the DNA regulatory regions as well as estimation of the potential role each TF binding motif would play. In the present example, we extended the previous model by including the nonlinear interactions among TF binding motifs. Furthermore, we evaluated the model-predicted role of the TF binding motifs by the promoter competition assay as described in Example II.

We modeled and evaluated the expression profiles of matrix metalloproteinase (MMP) genes in human synovial cells in response to mechanical shear. The family of MMPs are structurally-related zinc-binding proteolytic enzymes that play a critical role in tissue degradation and remodeling, inflammatory responses, and cell migration (12, 62, 63) Their expression is sensitive to environmental stimuli including mechanical shear (18). Shear-driven MMP regulation in synovium is of particular interest, since the motion of the synovial fluid during exercise induces shear forces and MMP production in the synovium causes degradation of joint tissue (59). The upstream regulatory regions of many MMP genes possess well-known TF binding motifs such as AP1, AP2, NFκB, and PEA3, but their role in mechanotransduction is largely unknown (7, 22) We applied uniform shear stress at 2 dyn/cm² to synovial cells in culture for 0-24 hours and determined the temporal mRNA expression profiles of a family of MMPs.

The complex expression pattern in response to shear was modeled by nonlinear formulation, and the model-based prediction of the role of the known TF binding motifs was evaluated by the promoter competition assay. Three modeling factors were TF binding motifs, 5′-end regulatory DNA regions, and a nonlinear parameter, λ (representing a degree of nonlinear interactions among TF binding motifs).

Materials and Methods for Example IV

Cell culture: MH7A synovial cell line (Riken Cell Bank, Japan (30) was used to determine the expression level of MMP mRNAs in response to mechanical stimuli for 0-24 hours.²⁴ The cells were fibroblast-like synoviocytes isolated from the knee of a patient with rheumatoid arthritis, and alteration of the MMP mRNA levels under mechanical shear was reported previously (18). Cells were cultured in RPMI1640 medium supplemented with 10% fetal calf serum and antibiotics. Using a Streamer Gold flow device (Flexcell International), fluid-driven uniform shear at 2 dyn/cm² was applied to the cultured cells for 1, 3, 6, 12, and 24 hours.

RT-PCR: Total RNA was isolated using RNeasy mini kits (Qiagen), and the isolated RNA was reverse-transcribed with random hexamers. Using a specific pair of primers for 13 MMPs (Table 4), the reverse-transcribed cDNA was amplified by semi-quantitative PCR. PCR was conducted in triplicate, ant the expression level of these MMPs was quantified using a gel scanner. The expression level was normalized into the value between “0” and “1” using glyceraldehype-3-phosphate dehydrogenase (GAPDH) as reference.

TABLE 4

| Gene | sense primer | antisense primer | cDNA size (bp) |
|---|---|---|---|
| MMP1 | 5′-CACAGCTTTCCTCCACTGCTGCTGC-3′ | 5′-GGCATGGTCCACATCTGCTCTTGGC-3′ | 396 |
| MMP2 | 5′GACAAGAACCAGATCACATAC-3′ | 5′GCCATGCTCCCAGCGGCCAAA-3′ | 181 |
| MMP3 | 5′-ATGAAGAGTCTTCCAATCCTACTGT-3′ | 5′-CATTATATCAGCCTCTCCTTCATAC-3 | 480 |
| MMP7 | 5′-GTGGTCACCTACAGGATCGT-3′ | 5′ACCATCCGTCCAGCGTTCAT-3′ | 202 |
| MMP8 | 5′AGCCAAATGAGGAAACTCTGG-3′ | 5′GATGCAACACTCCAGAGTTC-3′ | 283 |
| MMP9 | 5′GTGAGCTGGATAGCGCCACGC-3′ | 5′-CCGCGCTCCACAGTGCGAAGG-3′ | 201 |
| MMP10 | 5′-TCCTGACGTTGGTCACTTCAG-3′ | 5′-TCATACAGCCTGGAGAATGTG-3′ | 180 |
| MMP11 | 5′-GAGAAGACGGACCTCACCTAC-3′ | 5′-TGCCAGTACCTGGCGAAGTCG-3′ | 176 |
| MMP12 | 5′-CCACTGCTTCTGGAGCTCTT-3′ | 5′-GCGTAGTCAACATCCTCACG-3′ | 369 |
| MMP13 | 5′-TGGTGGTGATGAAGATGATTTGTCT-3′ | 5′-AGTTACATCGGACCAAACTTTGAAG-3′ | 376 |
| MMP14 | 5′-GCCATCGCTGCCATGCAGAAG-3′ | 5′-TTCATTATGTTGCCATTTAG-3′ | 180 |
| MMP15 | 5′-CAGAGATGCAGCGCTTCTACG-3′ | 5′-GATGGTGGTTGTTCCACTTCC-3′ | 180 |
| MMP16 | 5′-TGCGCTCTGCAGAGACCATGC-3′ | 5′-TCAATGCATATCGCTTTCGAC-3′ | 180 |
| MMP20 | 5′-GAAGCCTCGCTGTGGAGTTCC-3′ | 5′-CGGCGCTACTCCAGGCCTGCA-3′ | 170 |
| GAPDH | 5′-CCACCCATGGCAAATTCCATGGCA-3′ | 5′-TCTAGACGGCAGGTCAGGTCCACC-3′ | 598 |

Cluster analysis: A hierarchical clustering analysis was conducted by a custom-made computer program coded in Matlab (version 6.0, Mathworks Co., Ltd.). A family of MMPs was classified using Pearson's correlation coefficients among the expression profiles of 13 MMPs. A multidimensional scaling analysis (47) was conducted, and 13 MMPs were positioned in a 2D Euclidian space using SPSS statistics software (version 11.0, LEAD Technologies, Inc.).

Nonlinear least-square modeling: A linear least-square formulation was previously built in order to model the quantitative relationship between frequencies of the TF binding motifs and mRNA expression levels.¹⁶ However, the linear formulation was unable to model non-superimposable interactions among TF binding motifs. We developed a nonlinear least-square model with an assumption that the MMP mRNA level would be determined by nonlinearly additive interactions among multiple TF binding motifs:

z=h(x) (1)

where z represented an mRNA level of a family of MMP genes, x was defined as a cellular state at a specific time epoch under mechanical shear, and h was a nonlinear function that would describe the relationship between x and z.

The mathematical formulation utilized is set forth below:

A. Modeling of Nonlinear Interactions Among TF Binding Motifs

The principle in modeling nonlinear interactions among TF binding motifs is described using the simplified model:

z(x₁,x₂)=(f₁₀−f₀₀)x₁+(f₀₁−f₀₀)x₂+(f₁₁−f₁₀−f₀₁+f₀₀)x₁x₂+f₀₀

where an mRNA level of one gene, z, is represented by the state of two TF binding motifs, x₁ and x₂. The four coefficients are defined as f₀₀=z(0, 0), f₁₀=z(1, 0), f₀₁=z(0, 1), and f₁₁=z(1, 1) for z(x₁, x₂). We assume the nonlinear relationship, 1−f₀₀=(1−f₀₁)+f₀₁(1−f₁₀), which indicates that the role of two TF binding motifs is nonlinearly additive. From f₀₀=f₀₁f₁₀, z(x₁, x₂) is rewritten:

z(x₁, x₂)=h₁x₁+h₂x₂+λh₁h₂x₁x₂+1/λ

where h₁=f₁₀(1−f₀₁), h₂=f₀₁(1−f₁₀), and λ=1/f₀₁f₁₀. The Eq. (2) is a generalized form of the above relationship, where the value of λ is set constant for all genes. Although the described nonlinear model is a crude approximation of the complex molecular interactions, the model enables to include combinatorial interactions among TF binding motifs in the quadratic form.

B. Prediction of mRNA Expression Levels in the Promoter Competition Assay

We assume that introduction of exogenous DNA fragments consisting of a specific TF binding motif disturbs the specific component of the cellular state x corresponding to the transferred TF binding motif. Suppose that the cellular state, x, is perturbed by Δx in the promoter competition assay, then the perturbed mRNA expression would be modeled:

z_{perturbed}=h(x+Δx)

and the difference in the expression level would be:

Δz=z_{perturbed}−z

In order to estimate a contribution of Δx in a promoter competition assay to Δz, we used the following relationship:

Δzᵢ=αᵢ(∂h/∂xᵢ)

αᵢ=(∂h/∂xᵢ)^{T}Δz_{observed}/(∂h/∂xᵢ)^{T}(∂h/∂xᵢ)

where αᵢ was determined to minimize (Δz_{observed}−Δz_{modeled i})^{T}(Δz_{observed}−Δz_{modeled i}).

Based on mathematical formulation set forth above, the specific form of h, used in this study, was:

m m−1 m

zᵢ(x₁,x₂, . . . ,xₙ)=Σhᵢⱼxⱼ+λΣΣhᵢⱼhᵢₖxⱼxₖ+1/λ (2)

j=1 j=1 k=j+1

where zᵢ=mRNA expression level of the i-th MMPs (i=1, 2, . . . , n), xⱼ=cellular state of the j-th TF binding motif such as AP1, AP2, NFY, NFκB, PEA3, Sp1, and STAT (j=1, 2, . . . , m; m<n), hᵢⱼ=effectiveness of the j-th TF binding motif to the expression of the i-th MMP gene, and λ=nonlinear parameter indicating the degree of nonlinear interactions (λ>1).

The above nonlinear relationship between the expression level and the cellular state would enable us to model the molecular mechanism, for instance, observed in regulation of MMP2 mRNA: (i) blocking a single TF binding motifs (AP1, NFκB or PEA3) reduced the MMP2 mRNA level to ˜20% of the control level, and (ii) blocking simultaneously two TF binding motifs reduced it to ˜5% of the control level. Nonlinear formulation described here allowed biomedical engineers to model nonlinearly additive effects of these TF binding motifs. The value of hᵢⱼ was defined as the number of the j-th TF binding motifs on the 5′-flanking DNA sequences of the i-th MMP gene, and a SignalScan program for AP1, AP2, NFY, PEA3, and Sp1 (57) and a MatInspector program for NFκB and STAT¹⁷ were used to identify the known TF binding motifs. In modeling the mRNA expression levels, the vector x was optimally chosen using a nonlinear least-square procedure based on the Levenberg-Marquardt algorithm. We conducted Monte Carlo simulation and determined the model error for the scrambled data (randomly generated expression levels for 10,000 times).

Promoter Competition Assay:

In order to evaluate the described nonlinear model for the mRNA expression of MMPs, we conducted the promoter competition assay described in Example II. In brief, exogenous double-stranded DNA fragments consisting of a specific TF binding motif were transferred into cultured cells and the MMP expression was determined in the presence of the transferred DNA fragments (Table 5). Cells were incubated with exogenous DNA fragments at a concentration of 5 μM for 3 hours, and then the mRNA expression of MMPs was determined. Using the mathematical procedure described in Appendix B, the role of the TF binding motifs predicted by the nonlinear model was evaluated. DNA fragments with random sequences were used as control.

TABLE 5
Oligonucleotide sequences for promoter
competition assay

| Transcription factor | |
|---|---|
| binding site | Oligonucleotide sequences* |
| AP1 | 5′-TGACGTNTGASTCAGCATGC-3′ |
| AP2 | 5′-TGCAMKCCCSCNGGCGGACT-3′ |
| NFY | 5′-NCTGATTGGYTASY-3′ |
| NFκB | 5′-TGCAGGGGATYCCCGACT-3′ |
| PEA3 | 5′-TGACNCMGGAWGYNTCAG-3′ |
| Sp1 | 5′-GATCGGGGCGGGGCGATC-3′ |
| STAT | 5′-AGTCTTCCCRKAATGAC-3′ |
| random control | 5′-TGCAGACTCATGTAGCGT-3′ |

*K = T + G;
M = A + C;
N = A + C + G + T;
R = A + G;
S = C + G;
W = A + T;
Y = C + T.

### Results

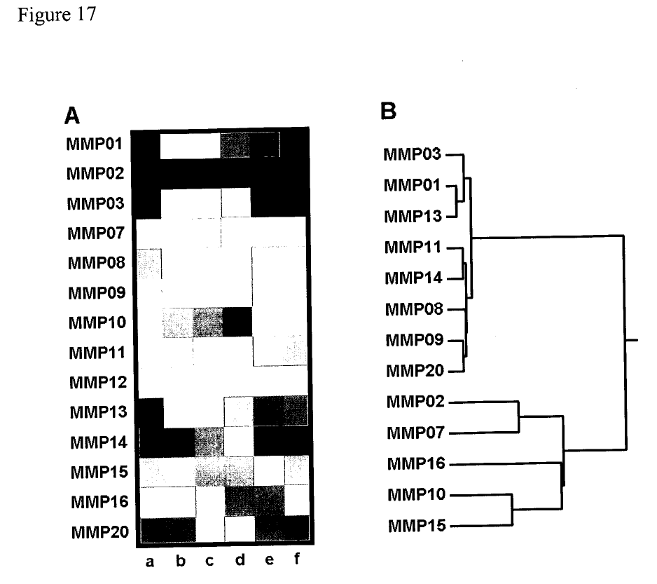

Heterogeneous MMP expression profile in response to mechanical shear: The temporal mRNA expression profile of 14 MMPs in human synovial cells was determined under shear stress at 2 dyn/cm² for 0, 1, 3, 6, 12, and 24 hours, and a hierarchical clustering dendogram was constructed (FIG. 17). MMPs were divided into two major clusters: 8 MMPs (MMP 3, 1, 13, 11, 14, 8, 9, and 20) that were downregulated by mechanical shear during 3-12 hours, and 5 MMPs (MMP 2, 7, 16, 10, and 15) that displayed upregulation at least one time epoch during the 24-hr shear treatment. MMP12 did not respond to shear and therefore was not included in the dendogram. The three collagenases, MMP1 (collagenase 1), MMP8 (collagenase 2), and MMP13 (collagenase 3), were clustered in the same group, while two gelatinases, MMP2 (gelatinase A) and MMP9 (gelatinase B), were not clustered together.

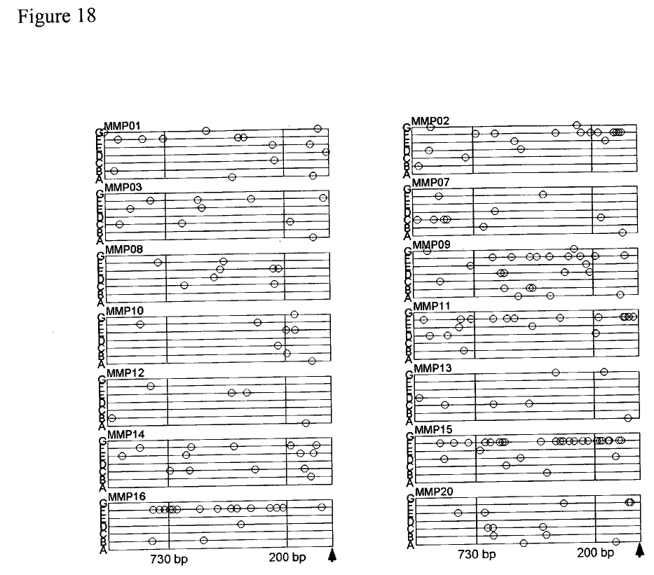

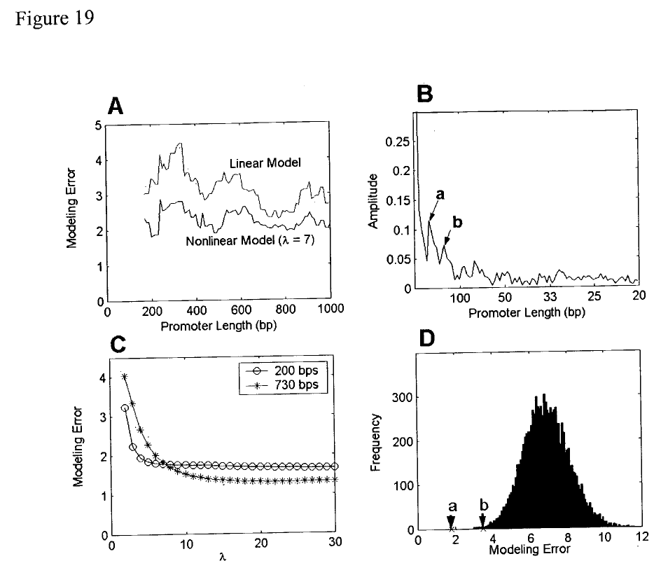

Parameters for mathematical formulation: In mathematical formulation, the three modeling factors considered were TF binding motifs, length of regulatory DNA regions, and the nonlinear parameter, λ. We focused on modeling the known TF binding motifs such as AP1, AP2, NFY, NFκB, PEA3, Sp1, and STAT on the 1000-bp upstream DNA sequences (FIG. 18). In order to evaluate the modeling factors for 84 data points (14 MMPs×6 time epochs), we determined the modeling error defined as a mean-square sum of differences between the observed expression level and the modeled expression level (FIG. 19). For the regulatory DNA region in the range of 180 to 1000 bp, the modeling error for the nonlinear model was always smaller than the modeling error for the linear model (FIG. 19A). A Fourier analysis revealed that the modeling error had a frequency of 151 and 302 bp, close to the size of nucleosome (FIG. 19B). The optimal length of the regulatory DNA sequences was 200 bp (λ=1 to 7) or 730 bp (λ=8 and above) (FIG. 19C). The mean-square model error was 1.8 (nonlinear model), 3.5 (linear model), and 6.9 (Monte Carlo simulation) (FIG. 19D).

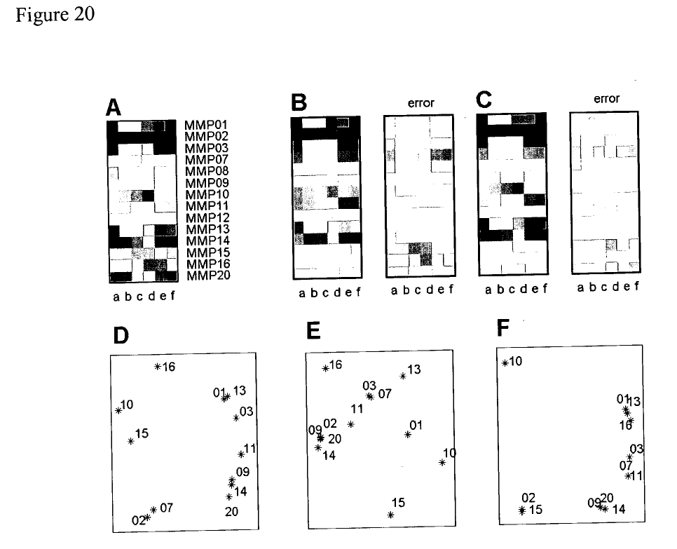

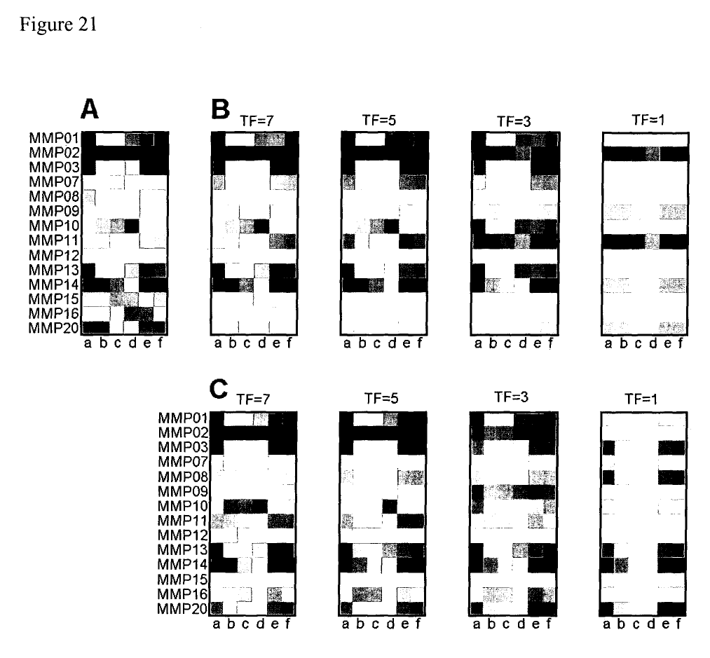

Characterization of linear and nonlinear models: Using the seven TF binding motifs on the 200-bp upstream regulatory DNA sequences, the observed expression pattern of MMPs was approximated by the linear formulation and the nonlinear formulation (FIG. 20A-20C). The multidimensional scaling analysis in 2D Euclidian space was conducted to visualize clustering of MMPs using the observed expression pattern and the linearly and nonlinearly modeled patterns (FIG. 20D-20F). Compared to the linear model, the overall positioning error was reduced in the nonlinear model where all MMPs except for MMP7 and MMP16 were clustered approximately at the expected position. Using a varying combination of TF binding motifs, we investigated a contribution of individual TF binding motifs on modeling error. The best combinations for seven, five, three, and one TF binding motif with the minimum mean-square modeling error are illustrated for two cases (200-bp and 730-bp upstream DNA sequences) (FIG. 21). The modeling error for the 730-bp promoter was 1.7, 2.0, 3.8, and 9.0 for 7, 5, 3, and 1 TF binding motif, respectively.

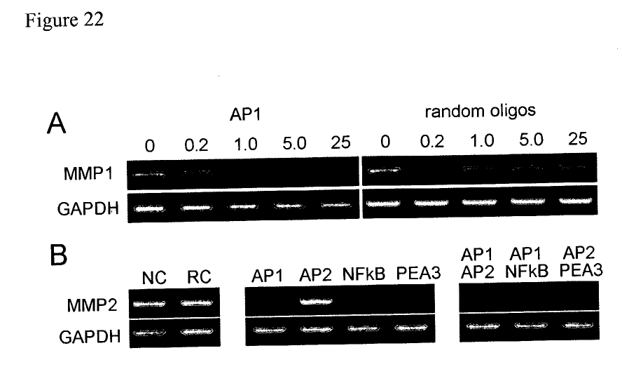

Nonlinear effects of multiple TF binding motifs: In order to evaluate the predicted role of the selected TF binding motifs, the promoter competition assay was conducted. Prior to a systematic analysis, we determined a proper dosage of transferred DNA fragments by monitoring MMP1 mRNA expression levels. The DNA fragments, consisting of an AP1 binding motif as well as no apparent binding motif (random DNA sequences for control), were transferred at 0.2, 1.0, 5.0, and 25 μM. Based on the dosage response, we determined to use the concentration of 5 μM hereafter (FIG. 22A).

Using MMP2 expression as a model case, a combinatorial effect of the DNA fragments containing AP1, AP2, NFκB, and/or PEA3 motifs was tested (FIG. 22B). The fragments with AP1 or NFκB motifs considerably reduced MMP2 expression, and the fragments with PEA3 motif made MMP2 expression undetectable in our PCR conditions. Competitive fragments with an AP2 motif, on the other hand, slightly upregulated MMP2 expression. These results indicated a stimulatory role of AP1, NFκB, and PEA3 and an inhibitory role of AP2. When a pair of DNA fragments with two different motifs was co-transferred, additive effects were observed. The combination of AP2 motif, with either an AP1 or PEA3 motif, partially suppressed the reduction seen by an AP1 or PEA3 motif alone. The combinatorial effect of AP1 and NFκB motifs was nonlinearly additive, supporting the principle of the nonlinear model.

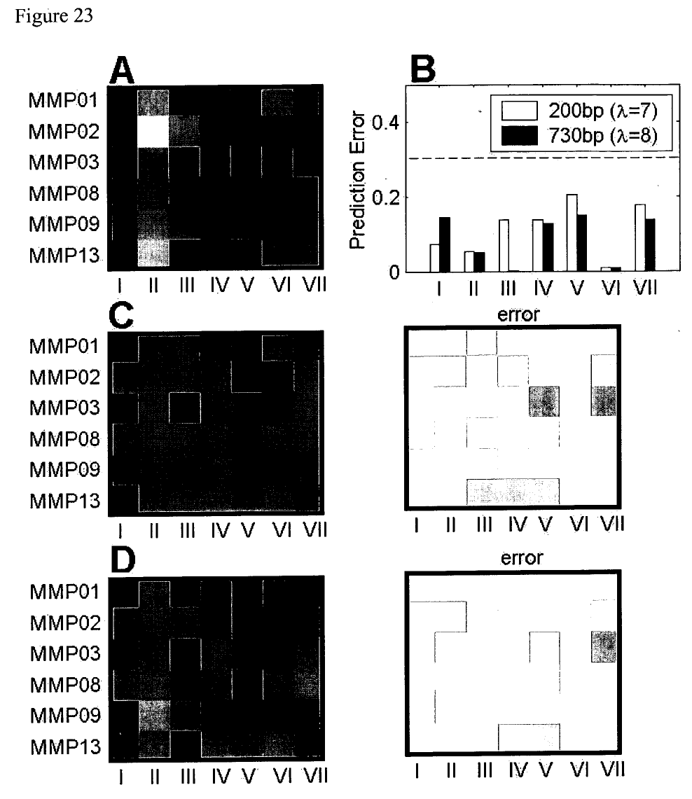

Promoter competition assay: In the promoter competition assay, we determined systematically the mRNA expression of 6 randomly selected MMPs (MMP1, 2, 3, 8, 9, and 13) in the presence of competitive TF binding motifs. The apparent functional significance of each TF binding motif is illustrated in FIG. 23A, where the rows from I to VII correspond to the assay for AP1, AP2, NFY, NFκB, PEA3, Sp1, and STAT motifs, respectively. For instance, column I represents the role of AP1 in the mRNA expression of the selected MMPs, and a strong stimulatory effect on MMP1, MMP3, and MMP13 is illustrated.

Based on the experimental cross-validation data, the prediction error of the nonlinear formulation for individual TF binding motifs was calculated (FIG. 23B). The mean-square sum of the prediction error was 4.8 (λ=7, 200-bp regulatory DNA region) and 3.7 (λ=8, 730-bp regulatory DNA region). These error values were smaller than the mean-square error of 12.8 in Monte Carlo simulation using the 10,000 randomly generated expression data, showing significant reduction of prediction error by the nonlinear model. The stimulatory role of Sp1 (column VI) was most accurately predicted among the selected TF binding motifs (FIGS. 23C and 23D).

Discussion

This example provides a novel model-based approach to extract the role of TF binding motifs in the temporal expression of a family of MMP genes under mechanical shear. The nonlinear mathematical model using the 5′-flanking DNA regions formulated the quantitative relationship between the distribution of the known TF binding motifs and their role in regulating the level of MMP transcripts. The role of the TF binding motifs predicted by the model was evaluated experimentally in the in vitro DNA transfer system. The nonlinear formulation included the interactions among the TF binding motifs and was able to approximate the observed MMP expression profiles more accurately than the linear formulation. The predicted stimulatory or inhibitory roles of the TF binding motifs, such as AP1, AP2, NFY, NFκB, PEA3, Sp1, and STAT, were in good agreement with the results obtained by the promoter competition assay.

A unique feature of the described model-based approach is its state-variable representation. The model is formulated using a set of state variables, and the activation level of state variables represents the cellular state under mechanical stimuli. We selected the biologically known TF binding motifs as state variables. With “m” TF binding motifs, the cellular state is defined by “m” state variables. In this sense, no fitting parameters are used in the model except for one parameter in the nonlinear model. With limited information on the nonlinear interactions among transcription factors, we did not add many parameters that would represent all possible interactions among transcription factors.

The nonlinear model is based on the results in the promoter competition assay, where the MMP2 mRNA level was decreased by ˜80% in the presence of either the AP1 competitor or the NFκB competitor. In the presence of both the AP1 and NFκB competitors, however, the MMP2 mRNA was decreased by ˜95% (not by ˜160%). The described nonlinear model was formulated to account for the following model: (i) Competitor A decreases the expression by ε, (ii) in the absence of Competitor A, Competitor B decreases the expression by ε, and (iii) in the presence of Competitor A, Competitor B decreases the expression by ε (1−ε).

The nonlinear model described herein provides improved modeling accuracy over the linear model and may be used in conjunction with linear model to predict transcription levels of target genes. The results in the promoter competition assay clearly indicate the nonlinearly additive role of a pair of TF binding motifs. In order to avoid assigning a set of adjusting parameters for varying combinations of TF binding motifs, we built the simplified nonlinear model that required one extra parameter, λ. Although λ may vary among TF binding motifs, the modeling error with λ>7 was relatively stable and the nonlinear formulation was able to approximate the expression profile with improved accuracy than the linear model in all cases we examined. Secondly, the described systems approach, in which the mathematical formulation is integrated into the in vitro evaluation system, (allows us to establish a feedback loop between model building and wet-lab experiments (51). Lastly, the results from the model would be useful to identify critical molecular targets in gene regulation and to develop a molecular strategy for tissue engineering.

The actual regulatory network in eukaryotic transcription is certainly more complex than the simplified mathematical model in this report. The proper selection of regulatory DNA regions, (43, 60) as well as the mathematical form of nonlinear interactions, requires a further understanding of the molecular mechanism underlying transcriptional regulation. Our results indicate that the 730-bp upstream regulatory DNA region can represent the expression pattern more accurately than the 200-bp regulatory region, but the results may depend on selection of TF binding motifs. Seven TF binding motifs chosen in this report are well known transcription factors. Since many MMPs are inducible by cytokines, oncogenic cellular transformation, physical stress, etc., other TFs in various signaling pathways, including unidentified regulatory DNA sequences, can be involved in MMP expression (50). Based on modeling error in the Monte Carlo simulation, both the linear and nonlinear models are capable of modeling, at least in part, the role of critical TF binding motifs (10).

All MMP genes share the similar function of degrading extracellular matrix, but our results clearly indicate that MMPs are regulated differently through a heterogeneous combination of TF binding motifs. We recognize that our in vitro results using a limited number of known TF binding motifs may represent an incomplete rendering of the normal physiological response to mechanical stimuli. Introduction of biologically meaningless state variables may result in a mere parameter fitting of the observed expression pattern. Nevertheless, the integrated usage of the nonlinear mathematical formulation and the biochemical evaluation system would provide a new dimension in the understanding of transcriptional mechanisms embedded in genomic DNA sequences, especially the orchestration of the shear stress responses among the known TF binding motifs.

### References

1. Collins, F. S., Patriano, A., Jordan, E., Chakravarti, A., Gesteland, R., Walters, L., the members of the DOE and NIH planning groups: New goals for the U.S. human genome project: 1998-2003. Science 282, 682-689 (1998)

2. International Human Genome Sequencing Consortium: Initial sequencing and analysis of the human genome. Nature 409, 860-921 (2001)

3. Ptashne, M. & Gann, A.: Transcriptional activation by recruitment. Nature 386, 569-577 (1997)

4. Prestridge, D. S.: SIGNAL SCAN: A computer program that scans DNA sequences for eukaryotic transcriptional elements. CABIOS 7, 203-206 (1991)

5. Quandt, K., Frech, K., Karas, H., Wingender, E. & Werner, T.: MatInd and MatInspector: new fast and versatile tools for detection of consensus matches in nucleotide sequence data. Nuc. Acids. Res. 23, 4878-4884 (1995)

6. Eisen, M. B., Spellman, P. T., Brown, P. O. & Botstein, D.: Cluster analysis and display of genome-wide expression patterns. Proc. Natl. Acad. Sci. U.S.A. 95, 14863-14868 (1998)

7. Alter, O., Brown, P. O. & Botstein, D.: Singular value decomposition for genome-wide expression data processing and modeling. Proc. Natl. Acad. Sci. U.S.A. 97, 10101-10106 (2000)

8. Tavazoie, S., Hughes, J. D., Campbell, M. J., Cho, R. J. & Church, G. M.: Systematic determination of genetic network architecture. Nature Genet. 22, 281-285 (1999)

9. Hughes, J. D., Estep, P. W., Tavazoie, S. & Church, G. M.: Computational identification of cis-regulatory elements associated with groups of functionally related genes in Saccharomyces cerevisiae. J. Mol. Biol. 296, 1205-1214 (2000)

10. Bussemaker, H. J., Li, H. & Siggia, E. D.: Regulatory element detection using correlation with expression. Nature Genet. 27, 167-171 (2001)

11. Konttinen, Y. T., Ainola, M., Valleala, H., Ma, J., Ida, H., Mandelin, J., Kinne, R. W., Santavirta, S., Sorsa, T., Lopez-Otin, C. & Takagi, M.: Analysis of 16 different matrix metalloproteinases (MMP-1 to MMP-20) in the synovial membrane: different profiles in trauma and rheumatoid arthritis. Ann. Rheum. Dis. 58, 691-697 (1999)

12. Shingleton, W. D., Hodes, D. J., Bzrick, P. & Cawston, T. E.: Collagenase: a key enzyme in collagen turnover. Biochem. Cell Biol. 74, 759-775 (1996)

13. Borden, P. & Heller, R. A.: Transcriptional control of matrix metalloproteinases and the tissue inhibitors of matrix metalloproteinases. Critical Rev. Eukaryotic Gene Expression 7, 159-178 (1997)

14. Arnett, F. C.: Rheumatoid arthritis, pp. 1492-1499. In Textbook of Medicine, Goldman, L., and Bennett, J. C. (eds.), W.B. Saunders Co., Philadelphia (2000)

15. Yoshihara, Y., Nakamura, H., Obata, K., Yamada, H., Hayakawa, T., Fujikawa, K. & Okada, Y.: Matrix metalloproteinases and tissue inhibitors of metalloproteinases in synovial fluids from patients with rheumatoid arthritis or osteoarthritis. Ann. Rheum. Dis. 59, 455-461 (2000)

16. Feldmann, M. & Maini, R. N.: The role of cytokines in the pathogenesis of rheumatoid arthritis. Rhematol. 38 (suppl. 2), 3-7 (1999)

17. Bunker, T. D., Reilly J., Baird, K. S. & Hamblen, D. L.: Expression of growth factors, cytokines and matrix metalloproteinases in frozen shoulder. J. Bone Joint Surgery, 82B, 768-773 (2000)

18. Sun, H. B. & Yokota, H.: Messenger-RNA expression of matrix metalloproteinases, tissue inhibitors of metalloproteinases, and transcription factors in rheumatic synovial cells under mechanical stimuli, Bone 28, 303-309 (2000)

19. Dinarello, C. A.: Interleukin-1. Cytokine Growth Factor Rev. 8, 253-265 (1997)

20. Stuuhl, K.: Fundamentally different logic of gene regulation in eukaryotes and prokaryotes. Cell 98, 1-4 (1999)

21. Mueller, J. M. & Pahl, H. L.: Assaying NF-κB and AP-1 DNA-binding and transcriptional activity. Methods Mol. Biol. 99, 205-216 (1999)

22. Benbow, U. & Brinckerhoff, C. E.: The AP-1 site and MMP gene regulation: What is all the fuss about? Matrix Biol. 15, 519-526 (1997)

23. Westermarck, J. & Kahari, V. M.: Regulation of matrix metalloproteinases expression in tumor invation. FASEB J. 13, 781-792 (1999)

24. Pendas, A. M., Santamaria, I., Alvarez, M. V., Pritchard, M. & Lopez-Otin, C.: Fine physical mapping of the human matrix metalloproteinase genes clustered on chromosome 11q22.3, Genomics 37, 264-265 (1996)

25. Gelb, A.: Applied Optimal Estimation, pp. 102-105. The MIT Press, Cambridge (1984)

26. Nowak, M. A. & Bangham, C. R. M.: Population dynamics of immune responses to persistent viruses. Science 272, 74-79 (1996)

27. Hammond, B. J.: Quantitative study of the control of HIV-1 gene expression. J. Theor. Biol. 163, 199-221 (1993)

28. Sun, H. B., Malacinski, G. M. & Yokota, H.: Promoter competition assay for analyzing gene regulation in joint tissue engineering (submitted)

29. Grande D. A., et al. Cartilage tissue engineering: current limitations and solutions: Clin. Orthopaedics Rlated Res. 367S, S176-S185 (1999)

30. Miyazawa K., Mori A. & Okudaira H.: Establishment and characterization of a novel human rheumatoid fibroblast-like synoviocyte line, MH7A, immortalized with SV40T antigen. J. Biochem. 124, 1153-1162 (1998)

31. Muller-Ladner U.: Molecular and cellular interactions in rheumatoid synovium. Curr. Opin. Rheumatol. 8, 210-220 (1996)

32. Jue D. M, Jeon K. I & Jeong J. Y. : Nuclear factor kappa B (NF-κB) pathway as a therapeutic target in rheumatoid arthritis. J. Korean Med. Sci. 14, 231-238 (1999)

33. Chen K. D., Li Y. S., Kim M., Li S., Yuan S., Chien S. & Shyy J. Y. J.: Mechanotransduction in response to shear stress. J. Biol. Chem. 274, 18393-18400 (1999)

34. Crooke S. T.: Progress in antisense technology: the end of the beginning. Methods Enzymol. 313, 3-45 (2000)

35. Agrawal S.: Importance of nucleotide sequence and chemical modifications of antisense oligonucleotides. Biochim. Biophy. Acta 1489, 53-68 (1999)

36. Ziauddin J. & Sabatini D. M.: Microarrays of cells expressing defined cDNAs. Nature 411, 107-110 (2001)

37. Reddi A. H.: Morphogenesis and tissue engineering of bone and cartilage: inductive signals, stem cells, and biomimetic biomaterials. Tissue Eng. 4, 351-359 (2000)

38. Iwakiri D. & Podolsky D. K.: Keratinocyte growth factor promotes goblet cell differentiation through regulation of goblet cell silencer inhibitor. Gastroenterol. 120, 1372-1380 (2001)

39. Lyons S. E., Shue B. C., Oates A. C., Zon L. I., & Liu P. P.: A novel myeloid-restricted zebrafish CCAAT/enhancer-binding protein with a potent transcriptional activation domain. Blood 97,2611-2617 (2001)

40. Arevalo-Silva C. A., Cao Y., Weng Y., Vacanti M., Rodriguez A., Vacanti C. A., & Eavey R. D.: The effect of fibroblast growth factor and transforming growth factor-beta on porcine chondrocytes and tissue-engineered autologous elastic cartilage. Tissue Eng. 7, 81-88 (2001).

41. Barnes, P. J., and Karin, M. 1997. Nuclear factor-κB—a pivotal transcription factor in chronic inflammatory diseases. New England J. Medicine 336:1066-1071.

42. Chin, J. E., Winterrowd, G. E., Krzesicki, R. F., and Sanders, M. E. 1990. Role of cytokines in inflammatory synovitis. Arthritis and Rheumatism 33:1776-1786.

43. Davuluri, R. V., Grosse, I., and Zhang, M. Q. 2001. Computational identification of promoters and first exons in the human genome. Nature Genetics 29:412-417.

44. de Jong, H. 2002. Modeling and simulating of genetic regulatory systems: a literature review. J. Computational Biol. 9:67-103.

45. DeRisi, J. L., and Iyer, V. R. 1999. Genomics and array technology. Current Opinion in Oncology 11:76-79.

46. Ding, G. J. F., Fischer, P. A., Boltz, R. C., Schmidt, J. A., Colaianne, J. J., Gough, A., Rubin, R. A., and Miller, D. K. 1998. Characterization and quantitation of NF-κB nuclear translocation induced by interleukin-1 and tumor necrosis factor-α. J. Biol. Chem. 273:28897-28905.

47. Khan, J., Simon, R., Bittnerr, M., Chen, Y., Leighton, S. B., Pohida, T., Smith, P. D., Jiang, Y., Gooden, G. C., Trent, J. M., and Meltzer, P. S. 1998. Gene expression profiling of Alveolar Rhabdomyosarcoma with cDNA microarrays. Cancer Res. 58:5009-5013.

48. Lockhart, D. J., and Winzeler, E. A. 2000. Genomnics, gene expression and DNA arrays. Nature 405:827-836.

49. Loeser, R. F., Sadiev, S., Tan, L., and Goldring, M. B. 2000. Integrin expression by primary and immortalized human chondrocytes: evidence of a differential role for α1β1 and α2β1 integrins in mediating chondrocyte adhesion to types II and VI collagen. Osteoarthritis and Cartilage 8:96-105.

50. Nagase, H., and Woessner, J. F. Jr. 1999. Matrix metalloproteinases. J. Biol. Chem. 274:21491-21494.

51. Ohler, U., and Niemann, H. 2001. Identification and analysis of eukaryotic promoters: recent computational approaches. Trends in Genetics 17:56-60.

52. Sun, H. B., and Yokota, H. 2002b. Reduction of cytokine-induced expression and activities of MMP-1 and MMP-13 by mechanical strain in MH7A rheumatoid synovial cells. Matrix Biol. 21:263-270.

53. van Helden, J., Andre, B., and Collado-Vides, J. 1998. Extracting regulatory sites form the upstream region of yeast genes by computational analysis of oligonucleotide frequencies. J. Mo. Biol. 281:827-842.

54. Vincenti, M. P., and Brinckerhoff, C. E. 2001. Early response genes induced in chodrocytes stimulated with the inflammatory cytokine interleukin-1β. Arthritis Res. 3:381-388.

55. Zhang, T., and Zhang, M. 2001. Promoter extraction from GenBank (PEG): automatic extraction of eukaryotic promoter sequences in large sets of genes. Bioinformatics 17:1232-1233.

56. Johannes, G., M. S. Carter, M. B. Eisen, P. O. Brown, and P. Sarnow. Identification of eukaryotic mRNAs that are translated at reduced cap binding complex eIF4F concentrations using a cDNA microarray. Proc.Natl.Acad.Sci. U.S.A 96: 13118-13123, 1999.

57. Prestridge, D. S. Computer software for eukaryotic promoter analysis. Methods Mol.Biol. 130: 265-295, 2000.

58. Scherf, U., D. T. Ross, M. Waltham, L. H. Smith, J. K. Lee, L. Tanabe, K. W. Kohn, W. C. Reinhold, T. G. Myers, D. T. Andrews, D. A. Scudiero, M. B. Eisen, E. A. Sausville, Y. Pommier, D. Botstein, P. O. Brown, and J. N. Weinstein. A gene expression database for the molecular pharmacology of cancer. Nat. Genet. 24: 236-244, 2000.

59. Schett, G., M. Tohidast-Akrad, G. Steiner, and J. Smolen. The stressed synovium. Arthritis Res. 3: 80-86, 2001.

60. Stormo, G. D. DNA binding sites: representation and discovery. Bioinformatics. 16: 16-23, 2000.

61. Venter, C. J. et al. The Sequence of the Human Genome. Science 271: 1304-1351, 2001.

62. Westermarck, J. and V. M. Kahari. Regulation of matrix metalloproteinase expression in tumor invasion. FASEB J. 13: 781-792, 1999.

63. Whittaker, M. and A. Ayscough. Matrix metalloproteinases and their inhibitors—current status and future challenges. Celltransmissions 17: 3-14, 2000.

The exemplary embodiments have been primarily described with reference to the figures which illustrate pertinent features of the embodiments. It should be appreciated that not all components or method steps of a complete implementation of a practical system are necessarily illustrated or described in detail. Rather, only those components or method steps necessary for a thorough understanding of the invention have been illustrated and described in detail. Actual implementations may utilize more steps or components or fewer steps or components. Thus, while certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for predicting an expression level of a target gene or gene family comprising:
a) selecting a target gene or gene family;
b) experimentally determining the number and type of cis-acting elements and mRNA expression levels of other genes within said target gene family to obtain a first data set; and
c) applying a PROBE algorithm to said data set, thereby generating the estimated expression level of said target gene as a function of the weighed frequencies of said cis-acting elements present in the 5′ untranslated regulatory region of said target gene.

2. The method of claim 1, wherein said cis-acting elements are selected from the group consisting of at least one of AP1, AP2, NFY, PEA3, Sp1, TFIID, NF-kappa B, STAT, GATA1, Oct-1 and TIE.

3. The method of claim 1, wherein said gene family is selected from the group consisting of matrix metalloproteinases, cytokines, hormones, IL-1 responsive genes, cyclins, growth factor receptors, growth factors, oncogenes, and transcription factors.

4. The method of claim 1, wherein said 5′regulatory region comprises at least 500 base pairs.

5. The method of claim 1, wherein said 5′regulatory region comprises at least 800 bases.

6. The method of claim 1, wherein said target genes are present on a microrray.

7. A method for predicting an expression level of a target gene or gene family in a particular cellular state, comprising:
a) selecting a target gene or gene family;
b) experimentally determining the number and type of cis-acting elements and mRNA expression levels of other genes within said target gene family in said cellular state relative to genes not in said cellular state to obtain a first data set; and
c) applying a non-linear model algorithm to said data set, thereby generating the estimated expression level of said target gene as a function of the cellular state and the weighed frequencies of said cis-acting elements present in the 5′ untranslated regulatory region of said target gene.

8. The method of claim 7, wherein said cis-acting elements are selected from the group consisting of at least one of AP1, AP2, NFY, PEA3, Sp1, TFIID, NF-kappa B, STAT, GATA1, Oct-1 and TIE.

9. The method of claim 7, wherein said gene family is selected from the group consisting of matrix metalloproteinases, cytokines, hormones, IL-1 responsive genes, cyclins, growth factor receptors, growth factors, oncogenes, and transcription factors.

10. The method of claim 7, wherein said 5′regulatory region comprises at least 500 base pairs.

11. The method of claim 7, wherein said 5′regulatory region comprises at least 800 bases.

12. The method of claim 7, wherein said target genes are matrix metallproteinases and said cellular state is induced by growing synovial cells under mechanical shear conditions.

13. The method of claim 7, wherein said target genes are present on a microarray.

14. A method for determining contribution of cis-acting elements to levels of mRNA expression of target genes, comprising:
a) providing a host cell population;
b) contacting said host cell with oligonucleotides encoding cis-acting element DNA, said cis-acting elements also being present in said target genes;
c) isolating mRNA from said host cells;
d) reverse transcribing said mRNA into cDNA;
e) performing polymerase chain reaction to amplify said cDNA and assessing alterations of expression levels of said target genes in the presence and absence of said oligonucleotide encoding cis-acting element DNA, altered mRNA expression levels indicating the presence of the oligonucleotide cis-acting element in the untranslated regulatory region of said target gene.

15. The method as claimed in claim 14, wherein said oligonucleotides are selected from the group consisting of CAGGC, CGCCC, CCGCC, CACCG, GCGCC, ATGGG, GGGAA and CCGCG.

16. The method of claim 14, wherein said target genes encode matrix metalloproteinases, said cis acting element is NF-kappa B and said oligonucleotide is TGCAGGGGATYCCCGACT.
